(19) United States
(12) Patent Application Publication
(10) Pub. No.: US 2006/0275841 A1
(43) Pub. Date: Dec. 7, 2006

(54) Assay method and apparatus with reduced sample matrix effects

(76) Inventors: Martin Blankfard, Falls Church, VA (US); Charles Quentin Davis, Frederick, MD (US); Richard Manteuffel, Laytonsville, MD (US); Manish S. Kochar, Rockville, MD (US); Daniel Curtis, Mt. Airy, MD (US); Qian Ning, Boyds, MD (US); Paul Macomber, Oak Hill, VA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/303,999

(22) Filed: Dec. 19, 2005

Related U.S. Application Data
(60) Provisional application No. 60/636,867, filed on Dec. 20, 2004.

(51) Int. Cl. G01N 33/53

(52) U.S. Cl. 435/7.5; 435/7.92

(57) ABSTRACT

The invention provides methods, kits, and apparatus for increasing the sensitivity of immunoassays performed on environmental samples by reducing sample matrix effects.

This application claims priority to U.S. Provisional Application No. 60/636,867, filed Dec. 20, 2004, all of which is incorporated by reference in its entirety.

### FIELD OF THE INVENTION

The present invention relates to assay methods, apparatus, and kits comprising an assay for detection of an analyte, such as an antigen or hapten in a sample. The analyte can be provided, for example, in a biological or air sample.

### BACKGROUND

In the medical, environmental, biodefense, and food safety communities, immunodiagnostic testing can provide a simple assessment and rapid identification of diseases and contaminants that are harmful to society. To prevent the occurrence of protracted illness and/or endemic disease, there is a need for simple confirmatory assays that provide qualitative, semi-quantitative, and quantitative detection of analytes, such as an antigen in a clinical specimen, soil or water sample, or food. In addition, due to the realization of the threat of national terrorism in recent years, many diagnostic tests are designed to be performed at satellite sites other than established laboratories.

Conventional immunoassay-based detection systems rely upon an antibody-antigen interaction, which requires the addition of multiple assay reagents in a sequential manner to produce a detectable event. Although generally reliable for positive identification, these assay and reagent preparation procedures can be affected by the sample matrix. For example, components in the sample may interfere with the ability of assay reagents to function properly.

Accordingly, there remains a need to develop new methods and apparatus for reliable and easy to use diagnostic assays that may be performed by non-technical or lay personnel with reduced sample matrix effects.

### SUMMARY

In some embodiments, the invention provides a method of analyzing a sample suspected of containing an analyte comprising:
(a) filtering the sample through a filter;
(b) adding a labeled binding partner specific to said analyte;
(c) adding an extraction buffer chosen from:
   (i) an extraction buffer that has a pH≧8 or pH≦6 and an osmolarity greater than or equal to 0.1 osmol/L and
   (ii) an extraction buffer that has an osmolarity greater than 1.1 osmol/L; and
(d) measuring the labeled binding partner in the resulting mixture,
   wherein steps (a) through (c) may be performed in any order and step (d) is performed last.

In certain embodiments, the invention provides a method of analyzing a sample suspected of containing an analyte comprising:
(a) filtering the sample through a filter;
(b) adding a first labeled binding partner specific to said analyte;
(c) adding a second binding partner specific to said analyte;
(d) adding an extraction buffer chosen from:
   (i) an extraction buffer that has a pH≧8 or pH≦6 and an osmolarity greater than or equal to 0.1 osmol/L and
   (ii) an extraction buffer that has an osmolarity greater than 1.1 osmol/L; and
(e) measuring the amount of the labeled first binding partner in complexes formed between the first binding partner, the analyte, and the second binding partner, wherein steps (a) through (d) may be performed in any order and step (e) is performed last.

In various embodiments, the invention provides a method of analyzing a sample suspected of containing an analyte comprising:
(a) filtering the sample through a filter;
(b) adding a labeled analog of the analyte;
(c) adding a binding partner specific to said analyte;
(d) adding an extraction buffer chosen from:
   (i) an extraction buffer that has a pH≧8 or pH≦6 and an osmolarity greater than or equal to 0.1 osmol/L and
   (ii) an extraction buffer that has an osmolarity greater than 1.1 osmol/L; and
(e) measuring the amount of the labeled analog of the analyte bound to the binding partner,
   wherein steps (a) through (d) may be performed in any order and step (e) is performed last.

In certain embodiments, the invention provides a kit for performing the above methods, comprising a filter and at least one reagent selected from (a) an extraction buffer that has a pH>8 or pH<6 and an osmolarity greater than or equal to 0.1 osmol/L and (b) an extraction buffer that has an osmolarity greater than 1.1 osmol/L.

In some embodiments, the invention provides an apparatus for use in analyzing sample suspected of containing one or more analytes comprising:
(a) a filter fluidically connected to a sampling device via a first one-way valve wherein the first one-way valve is oriented to allow flow from the sampling device and the filter;
(b) a waste line fluidically connected to a second one-way valve that is fluidically connected a point between the first one-way valve and the filter where the second one-way valve is oriented to allow flow from the filter to the waste line;
(c) a pump arranged to be able to reversibly drive liquid flow across the filter;
(d) a binding reagent container that holds a labeled binding partner specific for said one or more analytes;
(e) means for depositing filtrate from said filter into said binding reagent container; and
(f) means for measuring the labeled binding partner.

In various embodiments, the invention provides an apparatus for use in analyzing sample suspected of containing one or more analytes comprising:
(a) a filter fluidically connected to a sampling device;
(b) a pump arranged to be able to drive liquid flow across the filter;
(c) a binding reagent container that holds a labeled binding partner specific for said one or more analytes;
(d) means for depositing filtrate from said filter into said binding reagent container; and
(e) means for measuring the labeled binding partner.

In certain embodiments, the invention provides a sample filtering device comprising
(a) an upper container that is fluidically sealed from a lower container by a filter; and
(b) an opening where the lower container can be accessed without going through the upper container.

It is to be understood that both the foregoing general description and the following detailed description are exemplary and explanatory only and are not restrictive of the invention, as claimed. It will be appreciated that the section headings are provided as a guide to the reader and are not intended to limit the invention in any way.

The accompanying drawings illustrate embodiments of the invention.

### BRIEF DESCRIPTION OF THE DRAWINGS

FIG. 1 is a schematic representation of some exemplary embodiments of a reusable detection system.

FIG. 2 is a schematic representation of some exemplary embodiments of a disposable detection system.

FIGS. 3a and 3b are schematic representations of some exemplary embodiments of sampling and filtering portions of a detection system.

FIG. 4 is a schematic representation of some exemplary embodiments of sampling and filtering portions of a detection system.

FIG. 5 is a schematic representation of exemplary embodiments of disposable filters for a detection system.

FIG. 6 is a schematic representation of exemplary embodiments of disposable filters for a detection system.

FIG. 7 shows the suppressive effect of an environmental matrix (the MR matrix) on the signal to background ratio (S:B) of samples in a mouse IgG immunodetection assay. The symbols represent average measurements; the lines connect the average measurements.

FIG. 8 shows that performing the mouse IgG immunodetection according to the invention reduces the effect of an environmental matrix (the MR matrix) on the signal to background ratio (S:B). The symbols represent average measurements; the lines connect the average measurements.

FIG. 9 shows that filtering an environmental matrix sample comprising the MR matrix and ricin before use in an immunodetection assay reduces the effect of the environmental matrix on the signal. The symbols represent average measurements; the lines connect the average measurements.

FIG. 10 is a photomicrograph of an environmental matrix (the MR matrix) sample. The grid lines are separated by 40 μm.

FIG. 11 shows the deposition of superparamagnetic beads in an immunodetection assay performed on a sample comprising an environmental matrix (the MR matrix). The innermost circle is the portion of the working electrode that is directly visible to the photodetector, which has been removed to obtain these images. The brown specks that form one or more vertical bands are the superparamagnetic beads. (A) shows the deposition of beads when the sample was processed with a filter and an extraction buffer according to the invention. (B) is a photograph of bead deposition starting with the same environmental matrix-containing sample that was processed using the standard prior art method.

FIG. 12 shows the detection limits of an immunoassay for ricin in an environmental matrix (the MR matrix) according to the invention. The symbols represent average measurements; the curve is a dose response one site curve fit having the model formula

$$y = A + \frac{B - A}{1 + {( x / C )}^{D}} ,$$

wherein A, B, C, D are the fit parameters, y is the ECL signal value, and x is the concentration of ricin.

FIG. 13 shows the effect of an environmental matrix (the MR matrix) on the signal from an immunoassay for anthrax spores. The symbols represent average measurements; the lines connect the average measurements.

FIG. 14 shows the effect of borate extraction buffer on filtered samples comprising an environmental matrix (the MR matrix) and Bacillus globigii. The symbols represent average measurements; the lines connect the average measurements.

### DETAILED DESCRIPTION

Disclosed herein are methods, kits, and apparatus for use in detecting and/or quantifying an analyte by reducing sample matrix effects.

I. Definitions

The term “dry composition” means that the composition has a moisture content of less than or equal to 5% by weight, relative to the total weight of the composition.

The term “binding partner” means one or more substances that can bind specifically to an analyte. In general, specific binding is characterized by a relatively high affinity and a relatively low to moderate capacity. Typically, binding is considered specific when the affinity constant Kₐ is at least 10⁶ M⁻¹, at least 10⁷ M⁻¹, at least 10⁸ M⁻¹, or at least 10⁹ M⁻¹. A higher affinity constant indicates greater affinity, and thus typically greater specificity. For example, antibodies typically bind antigens with an affinity constant in the range of 10⁶ M⁻¹ to 10⁹ M⁻¹ or higher. Non-specific binding usually has a low affinity with a moderate to high capacity. Non-specific binding usually occurs when the affinity constant is below 106 M⁻¹.

The term “antibody” means a protein comprising one or more complementarity determining regions (CDRs) that retains antigen binding. In some embodiments, an antibody is an immunoglobulin or a part thereof, and encompasses any polypeptide (with or without further modification by sugar moieties (mono and polysaccharides)) comprising an antigen-binding site regardless of the source, method of production, or other characteristics. The term includes, for example, polyclonal, monoclonal, monospecific, polyspecific, humanized, single-chain, chimeric, synthetic, recombinant, hybrid, mutated, and CDR-grafted antibodies as well as fusion proteins. A part of an antibody can include any fragment which can bind antigen, including but not limited to, Fab, Fab′, F(ab′)₂, Facb, Fv, ScFv, Fd, V_{H}, and V_{L}.

The term “labeled binding partner” means a binding partner that comprises a label comprising an atom, moiety, functional group, or molecule capable of generating, modifying, or modulating a detectable signal.

The term “analyte” means any molecule, or aggregate of molecules, including a cell or a cellular component of a virus, archaea, plant animal, fungus, and/or bacteria, found in a sample. Also included in the scope of the term “analyte” are fragments of any molecule found in a sample. The definition further includes complexes comprising one or more of any of the examples set forth within this definition.

The term “analog of the analyte” refers to a substance that competes with the analyte of interest for binding to a binding partner. An analog of the analyte may be a known amount of the analyte of interest itself that is added to compete for binding to a specific binding partner with analyte of interest present in a sample. Examples of analogs of the analyte include azidothymidine (AZT), an analog of a nucleotide which binds to HIV reverse transcriptase, puromycin, an analog of the terminal aminoacyl-adenosine part of aminoacyl-tRNA, and methotrexate, an analog of tetrahydrofolate. Other analogs may be derivatives of the analyte of interest. The term “labeled analog of the analyte”, as used herein, is defined in an analogous manner to labeled binding partner.

The term “positive control” refers to a known amount of analyte or an analog of the analyte. Positive controls may be used to assess the proper instrument operation and/or sample measurement. Positive controls may be used to calibrate an instrument. Positive controls may be used as a reference to compare the signal level of the test sample with the signal level of the reference. Positive controls may also be used along with a mathematical function to relate signal levels with analyte concentrations, one use of which is to convert a signal measurement from a sample to an analyte concentration. The term “positive control” encompasses the common definition of both positive control and positive calibrator.

The term “assay positive control” refers to a composition used (a) to confirm successful measurement of a sample or (b) to convert a measured signal from a sample into a concentration of the tested analyte. Typically, an assay positive control comprises a positive control and the reagents used for a binding assay in order to simulate measurements from a sample that contains the analyte.

The term “sample” comprises liquids that may contain the analyte.

The term “liquid” comprises, in addition to the more traditional definition of liquid, colloids, suspensions, slurries, and dispersions of particles in a liquid wherein the particles have a sedimentation rate due to Earth's gravity of less than or equal to 1 mm/s.

The term “extraction buffer” refers to a composition used to reduce non-specific binding between an analyte or analog of an analyte and the sample matrix.

The term “fluidically connected” refers to a connection between two components wherein the connection allows a fluid to pass between the components.

The term “sample matrix” refers to everything in the sample with the exception of the analyte. The term “environmental matrix” refers to components of the sample matrix derived from the environment from which the sample is collected. For example, the sample matrix can contain reagents used for performing a detection assay that are not normally found in the sample environment. The term environmental matrix also refers to components of the sample matrix derived from the environment from which the sample is intended to be collected, although the analyte in the sample has been spiked in for assay development, simulations, or other purposes.

As used herein, the term “support” means any of the ways for immobilizing binding partners that are known in the art. Examples of support include, but are not limited to, membranes, beads, particles, electrodes, or even the walls or surfaces of a container.

The term “magnetizable bead” encompasses magnetic, paramagnetic, and superparamagnetic beads.

II. Methods

In certain embodiments, the invention relates methods that are used in binding assays for detecting or quantifying the amount of an analyte in a sample, wherein the effect of the sample matrix on the measurements is minimized.

A. Sample Matrix Effect on Analyte Detection

The sample matrix may effect the measurement of an analyte found in the sample in many ways. Without intending to be bound by any particular theory concerning the mechanism(s) through which the sample matrix may affect an assay, the matrix can, for example, reduce the rate of binding between the analyte and the binding partner by altering the sample's viscosity or otherwise changing the effective diffusion rate through the sample. In another scenario, the matrix can also non-specifically bind to the binding partner, reducing the number of available binding sites on the binding partner. The matrix can also non-specifically bind multiple analytes or multiple binding partners together, creating larger conglomerates that diffuse slower. The matrix can adhere to or settle on a support on which the binding partners are located, thus reducing the number of binding partners that are available to interact with the analyte. For supports that can move in the sample (e.g., beads), the matrix can adhere to multiple supports and create conglomerates that diffuse slower and/or settle to the bottom of the sample. The matrix can affect the amount of analyte bound at equilibrium. For example, large concentrations of the matrix can occupy a non-zero fraction of the binding partner's binding sites at equilibrium. If an enzyme is used to convert a substrate into a product, the matrix may reduce the rate of product formation.

The matrix can affect the measurement of the labeled binding partner. If the measurement process is a change in mass, the matrix, for example, can bind to the binding partner or to the analyte, effectively increasing the mass. If the measurement is responsive to agglutination, the matrix may cause agglutination in the absence of the analyte. If the measurement is luminescence, the matrix may scatter, absorb, or otherwise prevent the light from reaching the detector. If the measurement is luminescence, the matrix may create alternative non-radiative pathways from the excited state. If the measurement process is fluorescence, in addition to the luminescence mechanisms, the matrix may prevent the excitation light from being absorbed by the intended fluorescent compound or may auto-fluoresce. If the measurement process is chemiluminescence, in addition to the luminescence mechanisms, the matrix may reduce the rate of excited state formation through the mechanisms described in the preceding paragraph, which can interfere with the interaction of any two compounds whose interaction causes light to be emitted, as they can interfere with the interaction of an analyte with a binding partner. If the measurement process is electrochemiluminescence, in addition to the chemiluminescence mechanisms, the matrix can reduce the rate of energy transfer from the electrode to either an electrochemiluminescent compound or to an electrochemiluminescence coreactant, through, for example, the rate-altering mechanisms described in the preceding paragraph.

B. Reduction of Sample Matrix Effects

The effect of the matrix on the detection process can be reduced by either of two ways. First, the availability of the analyte for detection can be increased by changing solution properties that would prevent binding/association of the analyte with matrix components. Second, the components of the matrix that interfere with the detection process can be removed from the detection process. Several processes can be used to achieve this goal.

The analyte of interest can differ from the interfering matrix in several physical properties. These include, but are not limited to, differences in size, density, solubility, distribution of surface charges, or surface moieties. Separations based on size differences include use of filtration devices or passage through size exclusion columns. Separations based upon density differences include, but are not limited to, the use of centrifuges or gravity settling. Separations based upon solubility include, but are not limited to, two phase partitioning or solvent extraction into immiscible phases. Separations based upon surface charge differences include, but are not limited to, ion-exchange chromatography or flocculation and precipitation. Many of these separation methods are practical mainly in laboratory environments. Methods that are easy to use in non-laboratory situations include, but are not limited to, filtration and extraction solutions. In some embodiments, the invention uses an extraction buffer to release analyte from interfering matrix particles and/or filtration devices to remove these matrix particles from the sample.

1. Filtration

Filtration is one method for removing interfering components of the matrix from the detection process. In this approach, the barrier of the filter is used to retain particulate components that would interfere with the detection process. Particulate components of the matrix can interfere with some of the detection methodologies that require capture or deposition of labeled binding partners, labeled analog of the analyte, or beads on a detection surface. Additionally, removing these components decreases the amount of surface that could bind analyte. In some embodiments, the filter entrapped particulate components can be treated to release analyte for the detection process.

Filtration primarily separates components from solutions by presenting a physical barrier that excludes particles larger than a given size. There are many different methods in the filtration art to make barriers of these types, each method a function of the base material being manipulated. For example, metal wire is commonly used to make woven screens that can be used to catch extremely large particles, over 50 μm in size. To capture smaller air borne particles, smaller diameter metal wire screens can be used, but they have limitations due to impedance to air flow (pressure drop). Polymer based membranes are typically used to remove smaller particles from solutions. For example, the polymer nylon is used in a phase inversion casting process to make membranes that range from a 10 μm pore size rating, down to 0.1 μm pore size rating. Other polymer-based membranes (e.g. polyethersulphone, nitrocellulose, or cellulose acetate) are made by a solvent evaporation casting process. The filtration medium is generally incorporated into a holding device that allows the fluid of interest to pass through the filter barrier in a controlled manner. In some embodiments, the invention uses a filter containing filtration media using the polymer polyethersulphone (PES). In certain embodiments, the PES filter is encased in a plastic housing that can be (a) attached to a syringe, (b) part of a single use disposable device designed to ease robotic automation, or (c) part of a multiple-use disposable device designed to filter a plurality of samples.

Filtration primarily separates components from solutions by size. In most filters, the pathway through the filter is not a straight hole, but rather a twisted path. This makes describing filter pore size somewhat operational in nature, and gives rise to the term Pore Size Rating. In determining a filter's Pore Size Rating, filters are challenged with a known volume (or amount) of particles of a known size (known by a secondary means like microscopy, light scattering, or impedance measurements). Then, the amount of particles downstream of the filter is measured and compared to the amount of particles upstream of the filter, across multiple sizes of particles. When the ratio of downstream to upstream particles drops significantly below unity for a given size range of particles, the filter is said to have removal capacity for that size range. These ratios are typically described in logarithmic-based units of removal. For example, a filter rated at 5 μm will typically reduce the level of downstream particles greater than 5 μm by a ratio of 0.90 (90% removal or 1 log removal) to a ratio of 0.999 (99.9% removal: or 3 log removal). The pore size of the filter is chosen based many factors. The pore size should be large enough to pass the analyte, for example, anthrax spores are approximately 1 μm in size. The pore size should be small enough to block interfering components from the sample matrix. The pore size also affects the rate of fluid flow across the filter, with smaller pores generally creating larger resistances to flow.

In some embodiments, the invention can use a filter device with a pore size rating of 5 μm to remove interfering components of the sample matrix. In some embodiments, the invention can use a filter device with a pore size rating of 0.1, 0.2, 0.5, 1, 2, 3, 4, 7, 10, 15, 20, 50, or 100 μm to remove interfering components of the sample matrix. In some embodiments, the filter has a pore size rating less than or equal to about 100 μm and greater than or equal to about 10 μm to remove interfering components of the sample matrix. In some embodiments, the filter has a pore size rating less than or equal to about 10 μm and greater than or equal to about 1 μm to remove interfering components of the sample matrix. In some embodiments, the filter has a pore size rating less than or equal to about 1 μm and greater than or equal to about 0.1 μm to remove interfering components of the sample matrix. In some embodiments, the filter has a pore size rating less than or equal to about 0.1 μm and greater than or equal to about 0.02 μm to remove interfering components of the sample matrix.

Typically, particles smaller than the filter's pore size rating pass through a filter without hindrance, unless they are adsorbed to the filtration media. To prevent non-specific adsorption, filtration media can be surface-modified to reduce this type of interaction, e.g., by making the filter surface more wettable, i.e., more hydrophilic. It is generally believed that non-specific binding of analyte (that results in loss of recovery) is due to hydrophobic interactions, primarily through van der Waals interactions. For example, coating the filtration media polyethersulphone (PES) with hydrophilic compounds like glycerol increases the ability of water to wet the surface and reduces analyte loss.

Some surface treatments are more permanent and are considered surface grafts. Permanent surface modification of filtration media can be accomplished by many methods known to those skilled in the art. These methods include, but not limited to, free radical polymerization, ion beam initiated polymerization, ionizing radiation induced polymerization, plasma etching, and chemical coupling. These processes incorporate molecules with a significant number of hydroxyl groups that promote water hydration and reduce hydrophobic interactions. The specific method of surface modification depends primarily on the chemical nature of the filtration material used in the filter device. For example, ionizing radiation can be used to induce grafting of hydroxy-propyl-acrylate moieties onto nylon filtration media to render it hydrophilic and low protein binding. In some embodiments, the invention uses filtration media comprising the polymer polyethersulphone that was coated with glycerol to render the surface wettable with water and to reduce analyte loss.

Conversely, filters can have chemical moieties attached to the surface to specifically bind interfering components. The filtration media can be covalently coupled to molecules that have high affinity interactions with classes of molecules that are known interfere with the binding reaction (e.g., an immunoreaction) or the detection methodologies. For example, molecules like lectins, which will bind to surface groups on red blood cells, or ethylenediaminetetraacetic acid (EDTA), which bind metal ions that could interfere with the detection process, can be attached to the filtration media.

In various embodiments of the invention, filtration devices can be used to prepare samples for applications in which the number of analytes in the sample could vary, each analyte having specific preparation requirements. Some analytes are large and might be removed by small pore size filters. For example, analytes that are portions of bacteria might be removed by filters with the pore size ratings of sterilizing filters (0.2 μm), but would remain in the sample matrix after filtration using filters with pore size ratings of 5 μm. Conversely, the detection of small molecules like toxins can be enhanced by removing large particles by filters with pore size ratings of greater than or equal to 1 μm. Therefore, certain embodiments of the invention use several filtration devices in series to remove increasingly smaller particles, where samples for a specific analyte can be taken after the appropriate filtration device.

According to the invention, a sample can be filtered once or multiple times. In some applications, the sample's particulate load can be heavy and the layer of retained particles can be so large that a filtration device can be used only a single time. In other samples, the particle load can be low, such that multiple samples can be passed through the filtration device before the flow rate becomes too slow or the pressure drop becomes too high to be practical. In these cases, the filtration device can be replaced on some periodic interval that meets the needs and requirements of the sampling frequency. For example, if the particle load is low and if the frequency of a positive detection event is also low, then the filtration device can be changed monthly, weekly, daily, or sooner upon the occurrence of a positive detection event. The filter-changing interval can be set, for example, by the number of samples filtered, or by measuring the pressure drop across the filter or by measuring the flow rate through the filter.

2. Fluid Movement

According to the invention, multiple mechanisms can be used to drive the sample fluid through the filter. The fluid can be moved through the filter by gravity flow or by pressure, in which positive gauge pressure is applied upstream of the filter and/or negative gauge pressure (vacuum) is applied downstream of the filter.

The invention contemplates that a filter can be used multiple times for sample preparation when the particle retention layer can be reduced by a reverse flow (back flushing) process. In these embodiments, a reverse flow of fluids can be used to remove the particulates from the surfaces of the filtration devices and divert them to a waste collection system after the filtered sample has been taken for analysis.

3. Buffers

The recovery and detection of an analyte from a sample containing an interfering matrix can be increased by the use of appropriate extraction buffers. In some embodiments, the invention can use extraction buffers comprising, for example, sodium borate, sodium chloride, and nonionic detergents to increase analyte recovery. Alternative extraction buffers include sodium acetate, sodium malate, sodium oxalate, sodium citrate, sodium sulfate, sodium phosphate, as well as the potassium and lithium salts of borate, chloride, acetate, malate, oxalate, citrate, sulfate, and phosphate.

A portion of the analyte of interest can be associated with the sample matrix through low affinity, non-specific interactions. These types of interactions can include, e.g., both ionic and hydrophobic bonding. In some embodiments, the ionic interactions between an analyte and matrix particles can be reduced by increasing the overall ionic strength of the extraction buffer, so that the mobile solution ions pair with the ionic surface charges of the matrix, thereby promoting displacement of the analyte from the matrix particle surface. In various embodiments, the pH of the solution can be changed from neutral (i.e., pH 7) to either high pH or low pH to augment the ionic strength on reducing non-specific ionic interactions. Since most environmental matrix particles have a preponderance of negative surface charges, certain embodiments of the invention can use a high pH to ionize surface groups so that ions the extraction buffer can displace the analyte from the matrix particles. In some embodiments, the invention uses an extraction buffer with pH of 8.5 and at least 0.5 molar sodium chloride. In some embodiments, the invention uses an extraction buffer with a pH that is greater than or equal to 8.

In addition to ionic interactions, hydrophobic interactions can reduce analyte recovery. These types of interactions have been described as van der Waals types of interactions and can arise from the complex nature of water and hydrogen bonding. Ionic molecules can cause water molecules to form hydrogen bonded cage structures (clathrates) around the charge groups, which tend to organize water molecules and reduce the movement of water molecules. Molecules with polar groups can dissolve in water by forming hydrogen bond structures between the hydrogen of water and the polar group. Portions of molecules that have neither ionic charges nor polar groups can be considered hydrophobic, and these portions tend to be driven together by exclusion from hydration events. Molecules with hydrophobic portions can be driven together to engage in van der Waals interactions. In this way, the overall structure of water can be stabilized.

To increase analyte recovery due to low affinity non specific interaction with interfering matrix particles, certain embodiments of the invention can employ agents that cause a measured disruption of the water organization force. For example, hydrophobic interactions can be reduced by the use of surfactants/detergents and chaotropic ions, and uncharged chaotropic molecules. As used herein, surfactants and detergents are synonymous. Chaotropic molecules (charged as ions or uncharged) are molecules that tend to disrupt the organizing force and structure of water. In some embodiments, non-ionic surfactants (e.g., Tween® 20 (polyoxyethylene sorbitan monolaurate)) can be used to promote analyte recovery by binding the hydrophobic portion of the detergent molecule to the hydrophobic portions of the matrix and analyte. Uncharged chaotropic molecules (e.g. urea) can form solutions of sufficiently high concentrations that disrupt the hydrogen bonding structure of water. Various embodiments can also use borate, guanidine hydrochloride, guanidine thiocyanate or other chaotropic ions to promote the disruption of the hydrogen bonding structure of water. Borate ions are small and constrain the water molecule cage structures more than most ions. Phosphate and sulfate ions can also be used in the invention. Some embodiments of the invention use one or more cations such as Mg²⁺, Ca²⁺, Li⁺, Na⁺, or K⁺. One skilled in the art will appreciate that when using divalent or trivalent cations, additional unfavorable reactions may occur with some matrices. One skilled in the art will appreciate that, at a high concentration of chaotropic ions, the secondary and tertiary structures of protein molecules break down and high affinity interaction used in the immunoassay methods are disrupted. In some embodiments, the extraction buffer contains 0.1 M sodium borate (pH 8.5), 0.5 M sodium chloride, and 0.3% Tween® 20.

In various embodiments, the extraction buffer can have a variety of osmolarity ranges, as shown in Table 1. These ranges are exemplary only and the skilled artisan can recognize additional ranges applicable to the invention.

TABLE 1

| Lower Osmolarity Limit | Upper Osmolarity Limit |
|---|---|
| (osmol/L) | (osmol/L) |
| 1.1 | N/A |
| 1.2 | N/A |
| 1.5 | N/A |
| 2 | N/A |
| 2.5 | N/A |
| 1.1 | 11 |
| 1.2 | 11 |
| 1.1 | 6 |
| 1.2 | 6 |
| 1.1 | 3 |
| 1.2 | 3 |
| 1.1 | 2 |
| 1.2 | 2 |

N/A = There is no lower or upper limit.

The exemplary extraction buffers listed in Table 1 can optionally have a pH anywhere in the range from 2 to 11, from 3 to 10, from 4 to 9.5, from 5 to 8, from 6 to 8, from 7 to 10, from 8 to 10, from 8.5 to 9.7, and/or from 6.5 to 7.5.

In various embodiments, the extraction buffer can have a variety of pH and osmolarity combinations, as shown in Table 2. These combinations are exemplary only and the skilled artisan can recognize additional combinations applicable to the invention.

TABLE 2

| | | Lower | Upper |
|---|---|---|---|
| | | Osmolarity | Osmolarity |
| Lower | Upper | Limit | Limit |
| pH Limit | pH Limit | (osmol/L) | (osmol/L) |
| 8 | N/A | 0.05 | N/A |
| 8 | N/A | 0.1 | N/A |
| 8 | N/A | 0.3 | N/A |
| 8 | N/A | 0.6 | N/A |
| 8 | N/A | 0.8 | N/A |
| 8 | N/A | 1.0 | N/A |
| 8.4 | N/A | 0.05 | N/A |
| 8.4 | N/A | 0.1 | N/A |
| 8.4 | N/A | 0.3 | N/A |
| 8.4 | N/A | 0.6 | N/A |
| 8.4 | N/A | 0.8 | N/A |
| 8.4 | N/A | 1.0 | N/A |
| 8 | 9.7 | 0.05 | 4 |
| 8 | 9.7 | 0.1 | 3 |
| 8 | 9.7 | 0.4 | 2 |
| 8 | 9.7 | 0.8 | 2 |
| 8 | 9.7 | 1.2 | 2 |
| N/A | 6 | 0.05 | N/A |
| N/A | 6 | 0.1 | N/A |
| N/A | 6 | 0.3 | N/A |
| N/A | 6 | 0.6 | N/A |
| N/A | 6 | 0.8 | N/A |
| N/A | 6 | 1.0 | N/A |
| N/A | 5.6 | 0.05 | N/A |
| N/A | 5.6 | 0.1 | N/A |
| N/A | 5.6 | 0.3 | N/A |
| N/A | 5.6 | 0.6 | N/A |
| N/A | 5.6 | 0.8 | N/A |
| N/A | 5.6 | 1.0 | N/A |
| 4.5 | 6 | 0.05 | 4 |
| 4.5 | 6 | 0.1 | 3 |
| 4.5 | 6 | 0.4 | 2 |
| 4.5 | 6 | 0.8 | 2 |
| 4.5 | 6 | 1.2 | 2 |

N/A = There is no lower or upper limit.

The exemplary extraction buffers described in Tables 1 and 2 optionally comprise a surfactant. The particular surfactant chosen will reflect the chemical properties of the analyte and of sample matrix. For proteinaceous binding partners, the surfactant can be of the non ionic type, or in some embodiments, zwitterionic type. For nucleic acid binding partners, the surfactant can be of the nonionic, zwitterionic, anionic or cationic type.

Suitable nonionic detergents can be chosen from several categories, which include but limited to; alkyl glycosides, alkyl thioglycosides, alkyl maltosides, glucamides, polyoxyethylene alkyl ethers, polyoxyethylene polyoxypropylene block copolymers, polyoxyethylene polyoxyalkylene ethers, polyoxyalkylene alkyl ethers, sorbitan fatty acid esters, polyoxyethylene sorbitan fatty acid esters, polyoxyethylene sorbitol fatty acid esters, glycerol fatty acid esters, polyoxyethylene fatty acid esters, polyoxyethylene alkyl amines, alkyl dimethylphosphine oxides, alkyl dimethylamine oxides, and alkylol amides. Suitable zwitterionic detergents can be chosen from several categories, which include but limited to; alkyl betaines, alkyl sulfobetaines, alkyl aminopropanesulfonates, (Alky-dimethyl-amino)-propane-sulfonates, aryl aminopropanesulfonates, alkyl phosphocholine, aminopropylamine alkyl carboxylates, alkyl dimethyl glycines, alkyl phosphocholine, alkyl imido-dipropionate amine oxides, and poly alkyl dimethylamine propylamine carboxylates. Suitable anionic detergents can be chosen from several categories, which include but limited to; fatty acid salts, alkyl sulfates, bile acids, polyoxyethylene alkyl ether sulfates, alkylbenzene sulfonates, alkyl naphthalene sulfonates, dialkyl sulfosuccinates, alkyl diphenylene disulfonates, polyoxyethylene alkyl ether phosphates, polyoxyethylene alkyl ether sulfates, naphthalene sulfuric acid formaldehyde condensates, alkyl aminopropionates, and polycarboxylates. Suitable cationic detergents can be chosen from several categories, which include but limited to; alkyl amine salts, alkyl alkyl-quaternary ammonium salts, and alkyl aryl-quaternary ammonium salts.

In some embodiments, the extraction buffer can comprise a blocking agent. Any conventional blocking agents can be used. Suitable blocking agents are described, for example, in U.S. Pat. Nos. 5,807,752; 5,202,267; 5,399,500; 5,102,788; 4,931,385; 5,017,559; 4,818,686; 4,622,293; 4,468,469; and in CA 1,340,320; WO 97/05485; EP-A1-566,205; EP-A2-444,649; and EP-A1-165,669. Exemplary blocking agents include serum and serum albumins, such as animal serum (e.g., goat serum), bovine serum albumin (BSA), gelatin, biotin, and milk proteins (“blotto”). In some embodiments, the invention uses an extraction buffer comprising from 1 to 25 g/L BSA, from 1 to 10 g/L BSA, from 1 to 5 g/L BSA, or no BSA. In some embodiments, the invention uses an extraction buffer that contains 1.6 g/L BSA, 0.25 M HBO₃, 0.5 M NaCl, and 0.3% Tween-20 at a pH of 9.2. In some embodiments, the invention uses an extraction buffer that contains no BSA, 0.25 M HBO₃, 0.5 M NaCl, and 0.3% Tween-20 at a pH of 9.2.

C. Assay Formats

This invention can be used with any binding assay technique. See, for example, The Immunoassay Handbook, 3^{rd} Edition, Wild, Editor, Stockton Press, (2005) and Principles and Practice of Immunoassay, Price and Newman, Editors, Stockton Press, (1997). For convenience, a short description of some binding assay techniques follows.

Binding assay techniques can be subdivided in many ways. For example, some assays require a labeled binding partner for signal detection, while others generate a signal based on the interaction of the analyte and the binding partner, for example, measuring a mass change. Some assays do not use labeled binding partners, but instead use labeled analog of the analyte.

Some assays use two binding partners to create a sandwich assay where both binding partners bind specifically to the same analyte. In some embodiments, the two binding partners bind to different portions, e.g., different epitopes, of the analyte.

Some assays require a separation step to differentiate between a labeled binding partner that has bound an analyte and a labeled binding partner that has not bound an analyte. Some assays do not require a separation step, such as agglutination assays and assays wherein the label on the labeled binding partner is modified, activated, or deactivated directly or indirectly by the binding of the analyte. Some assays require a support to which a binding partner is attached. A support, separation, sandwich assay uses two binding partners—a first binding partner attached to the support, while a second binding partner is a labeled binding partner—to link the label to the support and afterwards washes the support to remove free labeled binding partner before measuring the label.

In some embodiments, the method of the invention detects an analyte by binding a labeled binding partner to the analyte. In some embodiments, the invention provides a method of analyzing a sample suspected of containing an analyte comprising:
(a) filtering the sample through a filter;
(b) adding a labeled binding partner specific to said analyte;
(c) adding an extraction buffer chosen from:
   (i) an extraction buffer that has a pH≧8 or pH≦6 and an osmolarity greater than or equal to 0.1 osmol/L and
   (ii) an extraction buffer that has an osmolarity greater than 1.1 osmol/L; and
(d) measuring the labeled binding partner in the resulting mixture,
   wherein steps (a) through (c) may be performed in any order and step (d) is performed last.

In some embodiments, the method of the invention detects an analyte by sandwiching the analyte between two binding partners, one of which is labeled. In certain embodiments, the invention provides a method of analyzing a sample suspected of containing an analyte comprising:
(a) filtering the sample through a filter;
(b) adding a first labeled binding partner specific to said analyte;
(c) adding a second binding partner specific to said analyte;
(d) adding an extraction buffer chosen from:
(i) an extraction buffer that has a pH≧8 or pH≦6 and an osmolarity greater than or equal to 0.1 osmol/L and
(ii) an extraction buffer that has an osmolarity greater than 1.1 osmol/L; and
(e) measuring the amount of the labeled first binding partner in complexes formed between the first binding partner, the analyte, and the second binding partner,
   wherein steps (a) through (d) may be performed in any order and step (e) is performed last.

In some embodiments, the method of the invention detects an analyte by using a labeled analyte analog to compete for binding to a binding partner. In various embodiments, the invention provides a method of analyzing a sample suspected of containing an analyte comprising:
(a) filtering the sample through a filter;
(b) adding a labeled analog of the analyte
(c) adding a binding partner specific to said analyte;
(d) adding an extraction buffer chosen from:
   (i) an extraction buffer that has a pH≧8 or pH≦6 and an osmolarity greater than or equal to 0.1 osmol/L and
   (ii) an extraction buffer that has an osmolarity greater than 1.1 osmol/L; and
(e) measuring the amount of the labeled analog of the analyte bound to the binding partner,
   wherein steps (a) through (d) may be performed in any order and step (e) is performed last.

The methods of the invention can be modified at various steps to reduce sample matrix effects. For example, in some embodiments, components in the sample matrix can be removed before a binding partner for the analyte to be detected contacts the sample. In some embodiments, the adding steps of the method may be performed simultaneously. In some embodiments, the adding steps of the method may be performed sequentially. In some embodiments, an extraction buffer is added to the sample, the sample is filtered, binding partner(s) are added one of which is labeled if more than one binding partner is used, and the labeled binding partner is detected. In some embodiments, the sample is filtered, an extraction buffer is added to the filtered sample, binding partner(s) are added one of which is labeled if more than one binding partner is used, and the labeled binding partner is detected. In some embodiments, an extraction buffer is added to the sample, the sample is filtered, a binding partner is added, a labeled analog of the analyte is added, and the labeled analog is detected. In some embodiments, the sample is filtered, an extraction buffer is added to the filtered sample, a binding partner is added, a labeled analog of the analyte is added, and the labeled analog is detected. In some embodiments, the sample is filtered; binding partner(s) are added, one of which is labeled if more than one binding partner is used; an extraction buffer is added; and the labeled binding partner is detected. In some embodiments, the sample is filtered, a binding partner is added, a labeled analog of the analyte is added, an extraction buffer is added, and the labeled analog is detected. Yet other embodiments involve methods that remove components in the sample matrix before a labeled binding partner contacts the sample. Some embodiments involve methods that remove components in the sample matrix before the sample contacts an electrode that can initiate electrochemiluminescence.

Some embodiments involve methods to reduce the sample matrix's ability to disturb the equilibrium or reaction rate between a binding partner for the analyte to be detected and said analyte. Some embodiments involve methods to reduce the sample matrix's ability to disturb the equilibrium or reaction rate between a labeled binding partner and the analyte to be detected.

Some embodiments involve methods to reduce the sample matrix's ability to affect the detection of the label on a labeling binding partner and/or a labeled analog of the analyte.

Some embodiments involve methods and apparatus to reduce the sample matrix's ability to affect electrochemiluminescence measurements. Some embodiments involve methods and apparatus to interface an apparatus able to perform binding assays with air sampling systems.

In some embodiments, multiple analytes are assayed simultaneously. In other embodiments, multiple analytes are assayed sequentially.

D. Samples and Analytes

A sample may be drawn from any source to be analyzed. For example, the sample may arise from body or other biological fluid, such as blood, plasma, serum, milk, semen, amniotic fluid, cerebral spinal fluid, sputum, tears, sweat, urine, or saliva. Alternatively, the sample may be a water sample obtained from a body of water, such as lake or river. The sample may also prepared by dissolving or suspending a sample in a liquid, such as water, a microbiological growth medium, or an aqueous buffer. The sample may be soil; talc; baby powder; food (e.g., fruits, vegetables, and/or meats); and/or human, ungulate, poultry, and/or other animal feces. These and other types of samples may be processed using mechanical treatment to remove the analyte from the matrix such as a Seward Stomacher® apparatus, sonication, blending, or any other physical method of transferring a solid into a liquid matrix. The sample source may be a surface swab; for example, a surface may be swabbed; the swab washed by a liquid; thereby transferring an analyte from the surface into the liquid. Swabs include sponges, paper towels, cotton and other absorbent tipped applicators. The sample source may be from air; for example, the air may be filtered; the filter washed by a liquid; thereby transferring an analyte from the air into the liquid. Air sampling devices can also bubble air directly into a liquid to transfer the analyte. For example, the air may be taken from inside a mailroom near mail handling machinery.

The methods of the invention can detect several types of analytes in a sample. Examples of analytes to which a binding partner can specifically bind include, but are not limited to, bacterial toxins, viruses, bacteria, proteins, hormones, DNA, RNA, drugs, antibiotics, nerve toxins, and metabolites thereof. Analytes also include fragments of any molecule found in a sample. In some embodiments, an analyte may be an organic compound, an organometallic compound or an inorganic compound. An analyte may be a nucleic acid (e.g., DNA, RNA, a plasmid, a vector, or an oligonucleotide), a protein (e.g., an antibody, an antigen, a receptor, a receptor ligand, or a peptide), a lipoprotein, a glycoprotein, a ribo- or deoxyribonucleoprotein, a peptide, a polysaccharide, a lipopolysaccharide, a lipid, a fatty acid, a vitamin, an amino acid, a pharmaceutical compound (e.g., tranquilizers, barbiturates, opiates, alcohols, tricyclic antidepressants, benzodiazepines, anti-virals, anti-fungals, antibiotics, steroids, cardiac glycosides, or a metabolite of any of the preceding), a hormone, a growth factor, an enzyme, a coenzyme, an apoenzyme, a hapten, a lectin, a substrate, a cellular metabolite, a cellular component or organelle (e.g., a membrane, a cell wall, a ribosome, a chromosome, a mitochondria, or a cytoskeleton component). In other embodiments, an analyte is a toxin, pesticide, herbicide, or an environmental pollutant.

Further examples of analytes include bacterial pathogens such as Aeromonas species such as Aeromonas hydrophila; Bacillus anthracis; Bacillus cereus; Botulinum neurotoxin producing species of Clostridium; Brucella abortus; Brucella melitensis; Brucella suis; Burkholderia mallei (formerly Pseudomonas mallei); Burkholderia pseudomallei (formerly Pseudomonas pseudomallei); Campylobacter jejuni; Chlamydia psittaci; Clostridium botulinum; Clostridium botulinum; Clostridium perfringens; Coccidioides immitis; Coccidioides posadasii; Cowdria ruminantium (Heartwater); Coxiella burnetii; Enterovirulent Escherichia coli group (EEC Group) such as Escherichia coli—enterotoxigenic (ETEC), Escherichia coli—enteropathogenic (EPEC), Escherichia coli—O157:H7 enterohemorrhagic (EHEC), and Escherichia coli—enteroinvasive (EIEC); Ehrlichia species such as Ehrlichia chaffeensis; Francisella tularensis; Legionella pneumophilia; Liberobacter africanus; Liberobacter asiaticus; Listeria monocytogenes; miscellaneous enterics such as Klebsiella, Enterobacter, Proteus, Citrobacter, Aerobacter, Providencia, and Serratia; Mycobacterium bovis; Mycobacterium tuberculosis; Mycoplasma capricolum; Mycoplasma mycoides ssp mycoides; Peronosclerospora philippinensis; Phakopsora pachyrhizi; Plesiomonas shigelloides; Ralstonia solanacearum race 3, biovar 2; Rickettsia prowazekii; Rickettsia rickettsii; Salmonella species; Schlerophthora rayssiae var zeae; Shigella species; Staphylococcus aureus; Staphylococcus aureus; Streptococcus; Synchytrium endobioticum; Vibrio cholerae non-O1; Vibrio cholerae O1; Vibrio parahaemolyticus and other Vibrios; Vibrio vulnificus; Xanthomonas oryzae; Xylella fastidiosa (citrus variegated chlorosis strain); Yersinia enterocolitica and Yersinia pseudotuberculosis; and Yersinia pestis.

Further examples of analytes include viruses such as African horse sickness virus; African swine fever virus; Akabane virus; Avian influenza virus (highly pathogenic); Bhanja virus; Blue tongue virus (exotic); Camel pox virus; Cercopithecine herpesvirus 1; Chikungunya virus; Classical swine fever virus; Coronavirus (SARS); Crimean-Congo hemorrhagic fever virus; Dengue viruses; Dugbe virus; Ebola viruses; Encephalitic viruses such as Eastern equine encephalitis virus, Japanese encephalitis virus, Murray Valley encephalitis, and Venezuelan equine encephalitis virus; Equine morbillivirus; Flexal virus; Foot and mouth disease virus; Germiston virus; Goat pox virus; Hantaan or other Hanta viruses; Hendra virus; Issyk-kul virus; Koutango virus; Lassa fever virus; Louping ill virus; Lumpy skin disease virus; Lymphocytic choriomeningitis virus; Malignant catarrhal fever virus (exotic); Marburg virus; Mayaro virus; Menangle virus; Monkeypox virus; Mucambo virus; Newcastle disease virus (VVND); Nipah Virus; Norwalk virus group; Oropouche virus; Orungo virus; Peste Des Petits Ruminants virus; Piry virus; Plum Pox Potyvirus; Poliovirus; Potato virus; Powassan virus; Rift Valley fever virus; Rinderpest virus; Rotavirus; Semliki Forest virus; Sheep pox virus; South American hemorrhagic fever viruses such as Flexal, Guanarito, Junin, Machupo, and Sabia; Spondweni virus; Swine vesicular disease virus; Tick-borne encephalitis complex (flavi) viruses such as Central European tick-borne encephalitis, Far Eastern tick-borne encephalitis, Russian spring and summer encephalitis, Kyasanur forest disease, and Omsk hemorrhagic fever; Variola major virus (Smallpox virus); Variola minor virus (Alastrim); Vesicular stomatitis virus (exotic); Wesselbron virus; West Nile virus; Yellow fever virus; viruses from the family papovaviridae, including polyomaviruses such as SV40, JC and BK and including papillomaviruses (e.g., HPV); parvoviruses (e.g., B19 and RA-1); and viruses from the family Picornaviridae, including rhinoviruses and Coxsackie B. Other viruses that can comprise a target nucleic acid sequence include species not mentioned above belonging to a family chosen from Adenoviridae, Arenaviridae, Arterivirus, Astroviridae, Baculoviridae, Badnavirus, Barnaviridae, Birnaviridae, Bromoviridae, Bunyaviridae, Caliciviridae, Capillovirus, Carla virus, Caulimovirus, Circoviridae, Closterovirus, Comoviridae, Coronaviridae, Corticoviridae, Cystoviridae, Deltavirus, Dianthovirus, Enamovirus, Filoviridae, Flaviviridae, Furovirus, Fuselloviridae, Geminiviridae, Hepadnaviridae, Herpesviridae, Hordeivirus, Hypoviridae, Idaeovirus, Inoviridae, Iridoviridae, Leviviridae, Lipothrixviridae, Luteovirus, Machlomovirus, Marafivirus, Microviridae, Myoviridae, Necrovirus, Nodaviridae, Orthomyxoviridae, Paramyxoviridae, Partitiviridae, Parvoviridae, Phycodnaviridae, Plasmaviridae, Podoviridae, Polydnaviridae, Potexvirus, Potyviridae, Poxyiridae, Reoviridae, Retroviridae, Rhabdoviridae, Rhizidiovirus, Sequiviridae, Siphoviridae, Sobemovirus, Tectiviridae, Tenuivirus, Tetraviridae, Tobamovirus, Tobravirus, Togaviridae, Tombusviridae, Totiviridae, Tymovirus, and Umbra virus.

Further examples of analytes include toxins such as Abrin; Aflatoxins; Botulinum neurotoxin; Ciguatera toxins; Clostridium perfringens epsilon toxin; Conotoxins; Diacetoxyscirpenol; Diphtheria toxin; Grayanotoxin; mushroom toxins such as amanitins, gyromitrin, and orellanine; Phytohaemagglutinin; Pyrrolizidine alkaloids; Ricin; Saxitoxin; shellfish toxins (paralytic, diarrheic, neurotoxic or amnesic) as saxitoxin, akadaic acid, dinophysis toxins, pectenotoxins, yessotoxins, brevetoxins, and domoic acid; Shigatoxins; Shiga-like ribosome inactivating proteins; snake toxins; Staphylococcal enterotoxins; T-2 toxin; and Tetrodotoxin.

Further examples of analytes include prion proteins such as the agents for transmissible spongiform encephalopathy diseases including scrapie, kuru, Creutzfeldt-Jakob disease (CJD), variant Creutzfeldt-Jakob Disease (vCJD), Chronic Wasting Disease, Fatal Familial Insomnia (FFI), Gerstmann-Straussler-Scheinker syndrome (GSS), and bovine spongiform encephalopathy (BSE or mad cow disease).

Further examples of analytes include parasitic protozoa and worms, such as: Acanthamoeba and other free-living amoebae; Anisakis sp. and other related worms Ascaris lumbricoides and Trichuris trichiura; Cryptosporidium parvum; Cyclospora cayetanensis; Diphyllobothrium species; Entamoeba histolytica; Eustrongylides species; Giardia lamblia; Nanophyetus species; Shistosoma species; Toxoplasma gondii; and Trichinella.

Further examples of analytes include fungi such as: Aspergillus species; Blastomyces dermatitidis; Candida; Coccidioides immitis; Coccidioides posadasii; Cryptococcus neoformans; Histoplasma capsulatum; Maize rust; Rice blast; Rice brown spot disease; Rye blast; Sporothrix schenckii; and wheat fungus.

Further examples of analytes include genetic elements, recombinant nucleic acids, and recombinant organisms, such as:
(1) nucleic acids (synthetic or naturally derived, contiguous or fragmented, in host chromosomes or in expression vectors) that can encode infectious and/or replication competent forms of any of the U.S. Health and Human Services (HHS) and U.S. Department of Agriculture (USDA) select agents (7 CFR § 331, 9° C.F.R. §121, and 42° C.F.R. §73).
(2) nucleic acids (synthetic or naturally derived) that encode the functional form(s) of any of the toxins listed if the nucleic acids:
   (i) are in a vector or host chromosome;
   (ii) can be expressed in vivo or in vitro; and/or
3) nucleic acid-protein complexes that participate in cellular regulatory events:
   (i) viral nucleic acid-protein complexes that are precursors to viral replication;
   (ii) RNA-protein complexes that modify RNA structure and regulate protein transcription events; or
   (iii) Nucleic acid-protein complexes that are regulated by hormones or secondary cell signaling molecules
(4) viruses, archaea, bacteria, fungi, and toxins that have been genetically modified.

Further examples of analytes include immune response molecules to the above-mentioned analyte examples such as IgA, IgD, IgE, IgG, and IgM.

E. Binding Partners

1. Types of Binding Partners

Examples of binding partners and their target analytes include, but are not limited to, complementary nucleic acid sequences (e.g., two DNA sequences which hybridize to each other; two RNA sequences which hybridize to each other; a DNA and an RNA sequence which hybridize to each other), an antibody and an antigen, a receptor and a ligand (e.g., TNF and TNFr-I, CD142 and Factor Vila, B7-2 and CD28, HIV-1 and CD4, ATR/TEM8 or CMG and the protective antigen moiety of anthrax toxin), an enzyme and a substrate, or a molecule and a binding protein (e.g., vitamin B12 and intrinsic factor, folate and folate binding protein). Further examples of binding partners include, but are not limited to, binding proteins, for example, vitamin B12 binding protein; DNA binding proteins such as the superclasses of basic domains, zinc-coordinating DNA binding domains, Helix-turn-helix, beta scaffold factors with minor groove contacts, other transcription factors.

In some embodiments, a binding partner is an antibody, which can be a monoclonal antibody or a set of polyclonal antibodies. A large number of monoclonal antibodies that bind to various analytes of interest are available, as exemplified by the listings in various catalogs, such as: Biochemicals and Reagents for Life Science Research, Sigma-Aldrich Co., P.O. Box 14508, St. Louis, Mo., 63178, (2002/2003); The Handbook—A Guide to Fluorescent Probes and Labeling Technologies, Tenth Edition, Invitrogen Co., Carlsbad, Calif. (2005); Life Science Products and Services to Accelerate Your Discovery, Invitrogen™ Life Technologies, 1600 Faraday Avenue, Carlsbad, Calif. 92008 (2003); Product Catalog, KPL, Two Cessna Court, Gaithersburg, Md. 20879 (2004 and 2005); and the Pierce Catalog, Pierce Chemical Company, P.O. Box 117, Rockford, Ill. 61105, (2005).

Other exemplary monoclonal antibodies include those that bind specifically to β-actin, DNA, digoxin, insulin, progesterone, human leukocyte markers, human interleukin-10, human interferon, human fibrinogen, p53, hepatitis B virus or a portion thereof, HIV virus or a portion thereof, tumor necrosis factor, or FK-506. In certain embodiments, the monoclonal antibody is chosen from antibodies that bind specifically to at least one of T4, T3, free T3, free T4, TSH (thyroid-stimulating hormone), thyroglobulin, TSH receptor, prolactin, LH (luteinizing hormone), FSH (follicle stimulating hormone), testosterone, progesterone, estradiol, hCG (human Chorionic Gondaotropin), hCG+β, SHBG (sex hormone-binding globulin), DHEA-S (dehydroepiandrosterone sulfate), hGH (human growth hormone), ACTH (adrenocorticotropic hormone), cortisol, insulin, ferritin, folate, RBC (red blood cell) folate, vitamin B12, vitamin D, C-peptide, troponin T, CK-MB (creatine kinase-myoglobin), myoglobin, pro-BNP (brain natriuretic peptide), HbsAg (hepatitis B surface antigen), HbeAg (hepatitis B e antigen), HIV antigen, HIV combined, H. pylori, β-CrossLaps, osteocalcin, PTH (parathyroid hormone), IgE, digoxin, digitoxin, AFP (α-fetoprotein), CEA (carcinoembryonic antigen), PSA (prostate specific antigen), free PSA, CA (cancer antigen) 19-9, CA 12-5, CA 72-4, cyfra 21-1, NSE (neuron specific enolase), S 100, P1NP (procollagen type 1 N-propeptide), PAPP-A (pregnancy-associated plasma protein-A), Lp-PLA2 (lipoprotein-associated phospholipase A2), sCD40L (soluble CD40 Ligand), IL 18, ricin, and Survivin.

Other exemplary monoclonal antibodies include those that bind specifically to the pathogens, viruses, toxins, protozoa, worms, prions, bacteria, archaea, and fungi described as exemplary analytes above.

Other exemplary monoclonal antibodies include anti-TPO (antithyroid peroxidase antibody), anti-HBc (Hepatitis B c antigen), anti-HBc/IgM, anti-HAV (hepatitis A virus), anti-HAV/IgM, anti-HCV (hepatitis C virus), anti-HIV, anti-HIV p-24, anti-rubella IgG, anti-rubella IgM, anti-toxoplasmosis IgG, anti-toxoplasmosis IgM, anti-CMV (cytomegalovirus) IgG, anti-CMV IgM, anti-HGV (hepatitis G virus), anti-Bacillus anthracis, and anti-HTLV (human T-lymphotropic virus).

2. Supports

In some embodiments, a binding partner can be immobilized on the support by any conventional means, e.g., adsorption, absorption, noncovalent binding, covalent binding with a crosslinking agent, or covalent linkage resulting from chemical activation of either or both of the support or the first binding partner. In some embodiments, the immobilization of the first binding partner by the support may be accomplished by using a binding pair. For example, one member of the binding pair, e.g., streptavidin or avidin, can be bound to the support and the other member of the same binding pair, e.g., biotin, can be bound to the first binding partner. Suitable means for immobilizing the first binding partner on the support are disclosed, for example, in the Pierce Catalog, Pierce Chemical Company, P.O. Box 117, Rockford, Ill. 61105, 1994.

Other examples of supports that can be used with a binding partner include, but are not limited to, membranes, beads, particles, electrodes, or even the walls or surfaces of a container. A support can comprise any material on which the binding partner is immobilized, such as nitrocellulose, polystyrene, polypropylene, polyvinyl chloride, ethylvinylacetate (EVA), glass, carbon, glassy carbon, carbon black, carbon nanotubes or fibrils, platinum, palladium, gold, silver, silver chloride, iridium, or rhodium.

In some embodiments, the support is a bead, such as a polystyrene bead or a magnetizable bead. In various embodiments, beads can have a number of different sizes, for example, 3 mm or larger, 3 mm or smaller, 1 mm or smaller, 0.1 mm or smaller, 100 μm or smaller, 10 μm or smaller, 5 μm or smaller, 2.8 μm, 1 μm, 1 am or smaller, 0.5 μm or smaller, 0.1 μm, or 0.1 μm or smaller. Bead size can also range from 0.01 μm to 5 mm, from 0.1 μm to 3 mm, from 1 μm to 3 μm, or from 2.8 μm to 5 μm. In certain embodiments, the beads are primarily spherical. In certain embodiments, the beads may not be spherical; for non-spherical beads, the above dimensions refer to the length of the longest straight line segment that can remain in the interior of the bead.

In certain embodiments, the support can have additional polymer coatings to assist in solubility, attachment of a binding partner, reduction of non specific binding of detection molecules, or attachment of controlling molecules integral for the detection process.

In other embodiments, the support is a microcentrifuge tube or at least one well of a multiwell plate.

3. Labeling of Binding Partner

In some embodiments, a binding partner can be labeled. A detectable label can be, for example, a fluorophore, a chromophore, a spin label, a radioisotope, an enzyme, Quantum Dot, beads, aminohexyl, pyrene, an antigenic determinant detectable by an antibody, a chemiluminescence moiety, or an electrochemiluminescence moiety (ECL moiety), haptens, luminescent labels, radioactive labels, quantum dots, beads, aminohexyl, pyrene, metal particles, spin labels, and dyes. Exemplary labels are disclosed in Hemmila, et al., J. Biochem. Biophys. Methods, vol. 26, pp. 283-290 (1993); Kakabakos, et al., Clin. Chem., vol. 38, pp. 338-342 (1992); Xu, et al., Clin. Chem., pp. 2038-2043 (1992); Hemmila, et al., Scand. J. Clin. Lab. Invest., vol. 48, pp. 389-400 (1988); Bioluminescence and Chemiluminescence Proceedings of the 9th International Symposium 1996, J. W. Hastings, et al., Eds., Wiley, New York, 1996; Bioluminescence and Chemiluminescence Instruments and Applications, Knox Van Dyre, Ed., CRC Press, Boca Raton, 1985; I. Hemmila, Applications of Fluorescence in Immunoassays, Chemical Analysis, Volume 117, Wiley, New York, 1991; and Blackburn, et al., Clin. Chem., vol. 37, p. 1534 (1991).

The labeled binding partner may be labeled with a fluorophore, such as one useful in fluorescence measurements, time-resolved fluorescence measurements, and/or fluorescence resonance energy transfer (FRET) measurements. Fluorophores that can be used in the method of the present invention include, but are not limited to, IR dyes, Dyomics dyes, phycoerythrine, cascade blue, Oregon green 488, pacific blue, rhodamine derivatives such as rhodamine green, 5(6)-carboxyfluorescein, cyanine dyes (i.e., Cy2, Cy3, Cy3.5, Cy5, Cy5.5, Cy 7) (diethyl-amino)coumarin, fluorescein (i.e., FITC), tetramethylrhodamine, lissamine, Texas Red, AMCA, TRITC, bodipy dyes, Alexa dyes, green fluorescent protein (GFP), GFP analogues, reef coral fluorescent proteins (RCFPs), RCFP analogues, and tandem dyes as described in U.S. Pat. Nos. 5,783,673; 5,272,257; and 5,171,843.

Enzyme labels that can be used in the present invention include, but are not limited to, soybean peroxidase, alkaline phosphatase, and horseradish peroxidase.

Radioisotopes that can be used in the present invention include, but are not limited to ¹³¹I, ³²P and ³⁵S

Chemiluminescent moieties that can be used in the present invention include, but are not limited to, acridinium, luminol, isoluminol, acridinium esters, acridinedione 1,2-dioxetanes, pyridopyridazines.

Further examples of labeled binding partners include binding partners that are labeled with a moiety, functional group, or molecule which is useful for generating a signal in an electrochemiluminescent (ECL) assay. The ECL moiety can be any compound that can be induced to repeatedly emit electromagnetic radiation by direct exposure to an electrochemical energy source. Such moieties, functional groups, or molecules are disclosed in U.S. Pat. Nos. 5,962,218; 5,945,344; 5,935,779; 5,858,676; 5,846,485; 5,811,236; 5,804,400; 5,798,083; 5,779,976; 5,770,459; 5,746,974; 5,744,367; 5,731,147; 5,720,922; 5,716,781; 5,714,089; 5,705,402; 5,700,427; 5,686,244; 5,679,519; 5,643,713; 5,641,623; 5,632,956; 5,624,637; 5,610,075; 5,597,910; 5,591,581; 5,543,112; 5,466,416; 5,453,356; 5,310,687; 5,296,191; 5,247,243; 5,238,808; 5,221,605; 5,189,549; 5,147,806; 5,093,268; 5,068,088; 5,061,445; and 6,808,939; Dong, L. et al., Anal. Biochem., vol. 236, pp. 344-347 (1996); Blohm, et al., Biomedical Products, vol. 21, No. 4: 60 (1996); Jameison, et al., Anal. Chem., vol. 68, pp. 1298-1302 (1996); Kibbey, et al., Nature Biotechnology, vol. 14, no. 3, pp. 259-260 (1996); Yu, et al., Applied and Environmental Microbiology, vol. 62, no. 2, pp. 587-592 (1996); Williams, American Biotechnology, p. 26 (January, 1996); Darsley, et al., Biomedical Products, vol. 21, no. 1, p. 133 (January, 1996); Kobrynski, et al., Clinical and Diagnostic Laboratory Immunology, vol. 3, no. 1, pp. 42-46 (January 1996); Williams, IVD Technology, pp. 28-31 (November, 1995); Deaver, Nature, vol. 377, pp. 758-760 (Oct. 26, 1995); Yu, et al., BioMedical Products, vol. 20, no. 10, p. 20 (October, 1995); Kibbey, et al., BioMedical Products, vol. 20, no. 9, p. 116 (September, 1995); Schutzbank, et al., Journal of Clinical Microbiology, vol. 33, pp. 2036-2041 (August, 1995); Stern, et al., Clinical Biochemistry, vol. 28, pp. 470-472 (August, 1995); Carlowicz, Clinical Laboratory News, vol. 21, no. 8, pp. 1-2 (August 1995); Gatto-Menking, et al., Biosensors & Bioelectronics, vol. 10, pp. 501-507 (July, 1995); Yu, et al., Journal of Bioluminescence and Chemiluminescence, vol. 10, pp. 239-245 (1995); Van Gemen, et al., Journal of Virology Methods, vol. 49, pp. 157-168 (1994); Yang, et al., Bio/Technology, vol. 12, pp. 193-194 (1994); Kenten, et al., Clinical Chemistry, vol. 38, pp. 873-879 (1992); Kenten, Non-radioactive Labeling and Detection of Biomolecules, Kessler, Ed., Springer, Berlin, pp. 175-179 (1992); Gudibande, et al., Journal of Molecular and Cellular Probes, vol. 6, pp. 495-503 (1992); Kenten, et al., Clinical Chemistry, vol. 37, pp. 1626-1632 (1991); Blackburn, et al., Clinical Chemistry, vol. 37, pp. 1534-1539 (1991), Electrogenerated Chemiluminescence, Bard, Editor, Marcel Dekker, (2004), and Knight, A and Greenway, G. Analyst 119:879-890 (1994).

In certain embodiments, the electrochemiluminescent moiety can comprise a metal, such as ruthenium, osmium, rhenium, iridium, rhodium, platinum, palladium, molybdenum, and technetium. In certain embodiments, the electrochemiluminescent moiety can comprise a rare earth metal, such as cerium, dysprosium, erbium, europium, gadolinium, holmium, lanthanum, lutetium, neodymium, praseodymium, promethium, terbium, thulium, and ytterbium. In certain embodiments, the electrochemiluminescent moiety can comprise a metal, such as ruthenium or osmium. In certain embodiments, the binding partner can be labeled with a ruthenium moiety, such as a tris-bipyridyl-ruthenium group such as ruthenium (II) tris-bipyridine and salts thereof ([Ru(bpy)₃]²⁺). Another exemplary ECL moiety can be [Ru(sulfo-bpy)₂ bpy]²⁺ whose structure is 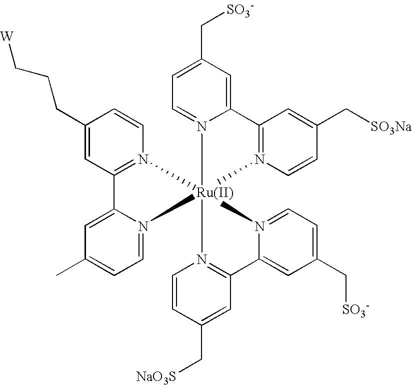
wherein W is a functional group attached to the ECL moiety that can react with a biological material, binding partner, enzyme substrate or other assay reagent so as to form a covalent linkage such as an NHS ester, an activated carboxyl, an amino group, a hydroxyl group, a carboxyl group, a hydrazide, a maleimide, or a phosphoramidite. The term [Ru(sulfo-bpy)₂ bpy]²⁺ also includes others salts of the shown structure.
III. Apparatuses

In some embodiments, the invention provides apparatuses for performing the methods described above.

In some embodiments, the invention provides an apparatus for use in analyzing sample suspected of containing one or more analytes comprising:
(a) a filter fluidically connected to a sampling device via a first one-way valve wherein the first one-way valve is oriented to allow flow from the sampling device and the filter;
(b) a waste line fluidically connected to a second one-way valve that is fluidically connected a point between the first one-way valve and the filter where the second one-way valve is oriented to allow flow from the filter to the waste line;
(c) a pump arranged to be able to reversibly drive liquid flow across the filter;
(d) a binding reagent container that holds a labeled binding partner specific for said one or more analytes;
(e) means for depositing filtrate from said filter into said binding reagent container; and
(f) means for measuring the labeled binding partner.

In various embodiments, the invention provides an apparatus for use in analyzing sample suspected of containing one or more analytes comprising:
(a) a filter fluidically connected to a sampling device;
(b) a pump arranged to be able to drive liquid flow across the filter;
(c) a binding reagent container that holds a labeled binding partner specific for said one or more analytes;
(d) means for depositing filtrate from said filter into said binding reagent container; and
(e) means for measuring the labeled binding partner.

In certain embodiments, the invention provides a sample filtering device comprising
(a) an upper container that is fluidically sealed from a lower container by a filter; and
(b) an opening where the lower container can be accessed without going through the upper container.

In some embodiments, the apparatus comprises an electrode can be used once or multiple times.

In some embodiments, the sampling device is an air sampler.

In some embodiments, the sample filtering device has radial symmetry. In other embodiments, the exterior of the sample filtering device does not have radial symmetry.

In embodiments that use luminescence to measure the labeled binding partner (e.g., electrochemiluminescence, fluorescence, or chemiluminescence), the apparatus can also include a photodetector for measuring the luminescent emission; for example, a photodiode, a CCD or CMOS sensor, and/or a photomultiplier tube.

1. Binding Reagent Containers

In some embodiments, the binding reagent container holds dry compositions. Examples of dry compositions include compositions that have a moisture content of less than or equal to 3% by weight, relative to the total weight of the composition and compositions that have a moisture content ranging from 1% to 3% by weight, relative to the total weight of the composition. In some embodiments, the binding reagent container comprises a moisture barrier that is sufficient to keep dry compositions within the container dry for at least 4 weeks at 25° C. and 95% relative humidity.

In some embodiments, the dry composition comprises compositions used for a binding assay and a positive control.

In certain embodiments, the binding reagent container can further comprise a lyophilization buffer. Lyophilization buffers are well known in the art and may contain phosphate buffer and, optionally, one or more cryoprotectants such as trehalose or sucrose.

In certain embodiments, the binding reagent container comprises an assay support that can be treated to block or reduce the nonspecific binding of the labeled second binding partner, analyte, or analog of the analyte to the support. Any conventional blocking agents can be used. Suitable blocking agents are described, for example, in U.S. Pat. Nos. 5,807,752; 5,202,267; 5,399,500; 5,102,788; 4,931,385; 5,017,559; 4,818,686; 4,622,293; 4,468,469; and in CA 1,340,320; WO 97/05485; EP-A1-566,205; EP-A2-444,649; and EP-A1-165,669. Exemplary blocking agents include serum and serum albumins, such as animal serum (e.g., goat serum), bovine serum albumin, gelatin, biotin, and milk proteins (“blotto”).

In some embodiments, the binding reagent container can be a multi-well plate that contains, for example, 24, 96, 384, 1536, or 6144 wells with each well able to contain one or more dry compositions. In certain embodiments, the multi-well plate, as used in an instrument, can have outside dimensions no larger than about the largest that is specified in the ANSI/SBS 20004 Microplate standards for footprint dimensions (ANSI/SBS 1-2004). The third dimension of the multi-well plate (i.e., the height), as used in an instrument, can have outside dimensions no larger than about 44 mm. In various embodiments, the container can be a tube that is less than or equal to about 9 mm in diameter, and less than or equal to about 40 mm tall. In some embodiments, the container can be a tube that has a maximum outside diameter of about 8.6 mm and a height of about 33.8 mm. In some embodiments, a two-dimensional array of binding reagent containers can be placed in a holder that is within the multi-well plate dimensions above. In various embodiments, the two-dimensional array of container in the holder can be 35 mm tall.

In some embodiments, the binding reagent container can be hermetically sealed. In some embodiments, the container can be sealed with an elastomeric, thermoset, or a thermoplastic material, such as EVA or Santoprene®, that has been pressed into the container's opening. In some embodiments, the binding reagent container can be sealed with a laminate comprising a metallic layer, such as a foil microplate seal. In various embodiments, the binding reagent container can be sealed with a laminate comprising a thermally modifiable layer, such as a laminate that can be heat-sealed to the container. In some embodiments, the binding reagent container can be sealed with a laminate comprising an adhesive layer that can bond the laminate to the container.

In some embodiments, the binding reagent container comprises at least one enclosure, such as one or more sealed enclosures (containers) inside a sealed bag. In some embodiments, the sealed bag can, for example, comprise polyethylene, polyester, aluminum, nickel, a trilaminate of polyester-foil-polyethylene, or a bilaminate of polyester-polyethylene. In some embodiments, a desiccant can be added between the innermost enclosure and the outermost enclosure. The desiccant can, for example, comprise calcium oxide, calcium chloride, calcium sulfate, silica, amorphous silicate, aluminosilicates, clay, activated alumina, zeolite, or molecular sieves. In some embodiments, a humidity indicator can be added between the innermost enclosure and the outermost enclosure. The humidity indicator can, for example, be used as an indication that the dry composition is still sufficiently dry that its stability has not been compromised. In some embodiments, the humidity indicator can be viewed through the outermost enclosure. In certain embodiments, the humidity indicator can be a card or disc wherein the humidity is indicated by a color change, such as one designed to meet the US military standard MS20003.

In some embodiments, the humidity barrier created by the container can be sufficient to keep the dry composition dry when the external conditions are 45° C. and 100% relative humidity for 10 days, 20 days, 40 days, 67 days, 3 months, 6 months, 12 months, 18 months, 24 months, or longer.

In some embodiments, the humidity barrier created by the container can be sufficient to keep the dry composition dry when the external conditions are 25° C. and 100% relative humidity for 1 day, 1 week, 1 month, 3 months, 6 months, 12 months, 18 months, 24 months, or longer.

In some embodiments, the humidity barrier created by the container can be sufficient to keep the dry composition dry when the external conditions are 4° C. and 100% relative humidity for 3 months, 6 months, 12 months, 18 months, 24 months, or longer.

2. Flow Cell-Based Biological Detection System

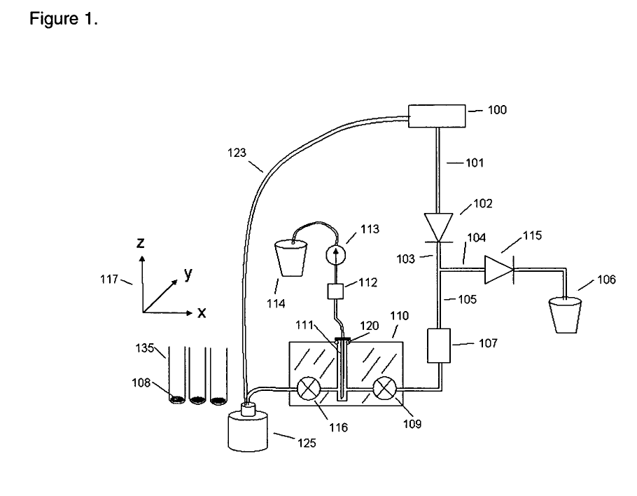

FIG. 1 is a schematic representation of an exemplary detection system, for example, a flow-cell based biological detection system. As depicted, overall operation of the detection system can be controlled by a computer system. Sample analysis occurs in flow cell 112, for example, a flow cell configured to measure radioactivity, optical absorbance, magnetic or magnetizable materials, light scattering, optical interference (i.e., interferometric measurements), refractive index changes, surface plasmon resonance, and/or luminescence (e.g., fluorescence, chemiluminescence and electrochemiluminescence). According to one aspect, the flow cell 112 can be adapted for conducting electrochemiluminescence measurements. Exemplary electrochemiluminescence flow cells and methods for their use are disclosed in U.S. Pat. No. 6,200,531. The operation of flow cell 112 can be controlled by a computer system, which can also receive assay data from the flow cell 112 and carry out data analysis. Flow cell 112 can be configured to detect the difference between liquid and air, which can be used to ensure sample in appropriately aspirated.

The exemplary flow-cell based biological detection system can comprise a fluid handling station for introducing one or more reagents and/or one or more samples that can include gases and liquids. FIG. 1 depicts a fluid handling station 110 that can comprise flow control valves 109 and 116 as well as a manifold for accepting pipettor 111. Additional flow control valves can also be present, as well as reagent/gas detectors.

The pipettor 111 can be able to form a air-tight seal into the fluid handing station 110 using an o-ring 120 arranged on a sealing surface of the manifold and a sealing surface of the pipettor (e.g., a collar, flange, or the like). As depicted, the fluid handling manifold sealing surface can be located away from the reagent input lines (e.g., above the reagent lines' aspiration chamber entry points). Additionally, one or more of the reagent entry points can be positioned at predetermined heights within the aspiration chamber. For example, as depicted, the liquid reagent lines can be positioned beneath a gas reagent line to preclude contamination of the gas line. Reagent aspiration can be controlled by coordinating the selective actuation of one or more of the flow control valves 109, 116 with the proper positioning of the pipettor and activation of the pump 113 so as to draw the reagents from the selected reagent bottle 125, from the filter 107, or from possible additional bottles or air via flow control valves not shown.

The detection system can be capable of precisely and accurately positioning the pipettor 111 and one or more binding reagent containers 135 so that the pipettor can be directed to aspirate/dispense fluids from a container and/or fluid handling station. The containers can have dry or wet binding reagents 108. The relative positioning of pipettor 111 and binding reagent container(s) 135 can be effected by a three-dimensional positioning system 117. The three directions of motion can be very close to mutually perpendicular, perhaps only having fabrication-related perturbations from perpendicularity, or may be distinctly non-perpendicular, perhaps due the lack of a requirement to move over all points in a rectangular box. Alternatively, the positioning system can be based on alternative coordinate systems (e.g., one dimensional, two dimensional, polar coordinates, etc.).

In some embodiments, sample collection device 100 can collect, for example, a wet-concentrated air sample. An exemplary sample collection device is the SpinCon® advanced air sampler by Sceptor Industries (Kansas City, Mo.). In certain embodiments, sample collection device 100 can be an open cup or repository that samples can be placed into, for example, by hand. Optionally, the sample collection device can use an extraction buffer located in reagent bottle 125 via tubing 123. Alternatively, a different liquid can be used by sample collection device 100 that can be located in the device or externally in a container. Optionally, the tubing 101 from sample collection device 100 can be vented to atmosphere within the device. Pump 113 can aspirate a filtrate from the sample collection device 100 via pipettor 111, flow control valve 109, filter 107, tubing 105, tubing 103, one-way valve 102, and tubing 101. Sample collection device 100 can be emptied of excess sample liquid by using pump 113 in an alternating fashion to pull liquids into tubing 105 via one-way valve 102 and push liquids from tubing 105 to waste 106 via tubing 104 and one-way valve 115. Filter 107 can be back-flushed or at least partially cleared by using pump 113 in an alternating fashion to draw extraction buffer (for example) into at least pipettor 111 via flow control valve 116 and to push extraction buffer through filter 107 and out to waste 106 via one-way valve 115.

Sample from collection device 100 can be filtered through filter 107 and drawn into pipettor 111. Sample can then be dispensed into binding reagent container 135. Extraction buffer from reagent bottle 125 or other reagent can additionally be dispensed into binding reagent container 135. Binding reactions between a binding reagent and analyte found in the sample can then occur. After a time interval, additional extraction buffer from reagent bottle 125 or other reagent can be dispensed into binding reagent container 135. After another time interval, the reaction mixture can be aspirated into the flow cell 112 for measurement. Depending on the measurement technology, additional reagents may be needed in the flow cell before the measurement can take place. For example, for electrochemiluminescence measurements, a coreactant is typically added to the flow cell while performing a free-bound separation on the reaction mixture in the flow cell. After measurement, the sample can be dispensed to waste.

3. Detection System Without a Flow Cell

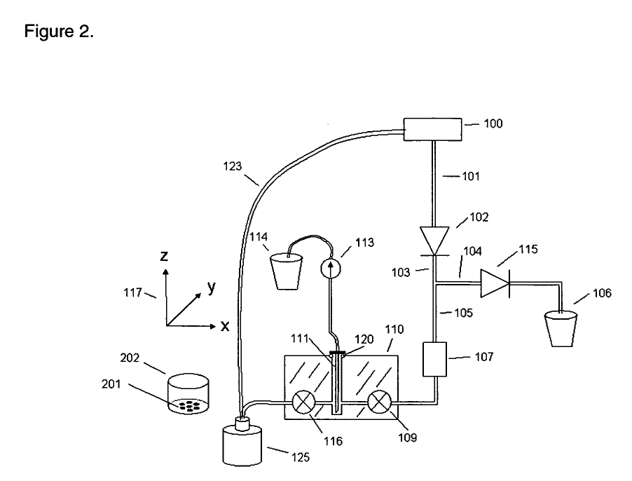

FIG. 2 demonstrates some embodiments of the detection system that do not use a flow cell. These embodiments can retain the ability to detect liquid versus air or other means of ensuring sample was aspirated. Speaking only of the distinctions between these embodiments and the flow cell embodiments described above, in these embodiments, binding reagent container 202 can hold in liquid or dry form binding partners as well as other materials needed for the measurement. For example, for electrochemiluminescence measurements, binding reagent container 202 can comprise an electrode to initiate electrochemiluminescence. For colorimetric measurements, binding reagent container 202 can contain an optical path to measure optical characteristics of the sample in situ. In some embodiments, binding reagent container 202 can hold binding partners for multiple analytes. These binding partners can be spatially separated as shown in the figure as 201, or can be co-located, depending at least in part on how the measurement technology distinguishes among the labeled binding partners. Pipettor 111 can pipette the sample into the container, the extraction buffer, and/or other reagents necessary for the measurement. Pipettor 111 can also be used in an alternating fashion to wash or exchange the contents of binding reagent container 202.

4. Alternative Configurations

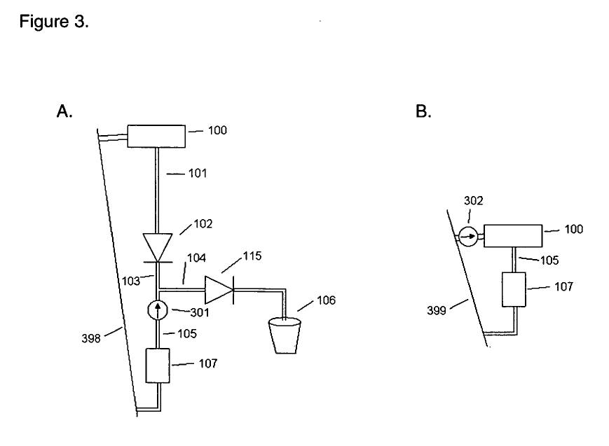

FIG. 3 demonstrates some embodiments that employ alternate architectures to obtain the sample. As shown in FIG. 3a, a pump 301 can be placed before filter 107. In this location, pump 301 can create large pressures across filter 107 to enable more expeditious filtering of the sample. Pump 301 can also include a pressure-monitoring device to ensure that excessive pressures are not created. Other features such as sending excess sample to waste and back flushing filter 107 can be retained. For brevity, diagonal line 398 shows where the apparatus of either FIG. 1 or FIG. 2 can be connected to this system. FIG. 3b shows an exemplary embodiment where sample collection device 100 does not have a vent that is always open at the pressures used. Pump 302 can push liquid through sample collection device 100 and filter 107. Pump 302 can also include a pressure-monitoring device to ensure that excessive pressures are not created. For brevity, diagonal line 399 shows where the apparatus of either FIG. 1 or FIG. 2 can be connected to this system.

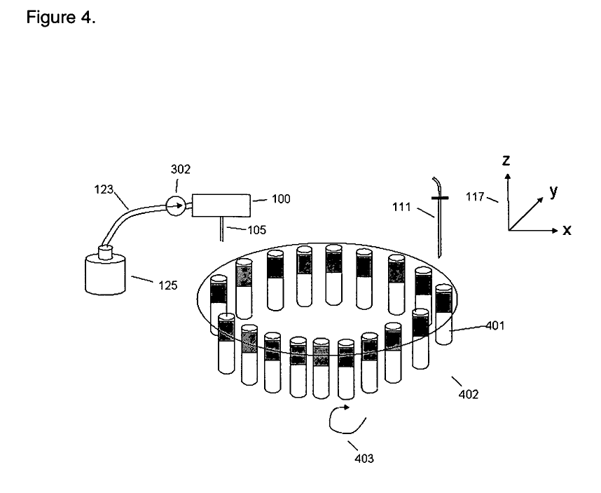

As shown in FIG. 4, some embodiments tubing 105 is not at all times in fluidic contact with the filter. As depicted here, sample collection device 100 can be optically connected via pump 302 and tubing 123 to extraction buffer located in reagent bottle 125. Alternatively, a different liquid can be used by sample collection device 100. Pump 302 can dispense sample into container 401 located in container-holder 402. Via motion control 403 that moves container-holder 402 and motion control 117 that moves pipettor 111, the sample can then be aspirated from container 401 into, for example, binding reagent container 135 or binding reagent container 202 for binding with the binding partner and eventual measurement. In some embodiments, sample can be placed in container 401 by hand or an alternate piece of robotic integration. While FIG. 4 shows a circular arrangement of containers 401 and container-holder 402, other shapes (e.g., a rectangular array or a linear array) are also contemplated.

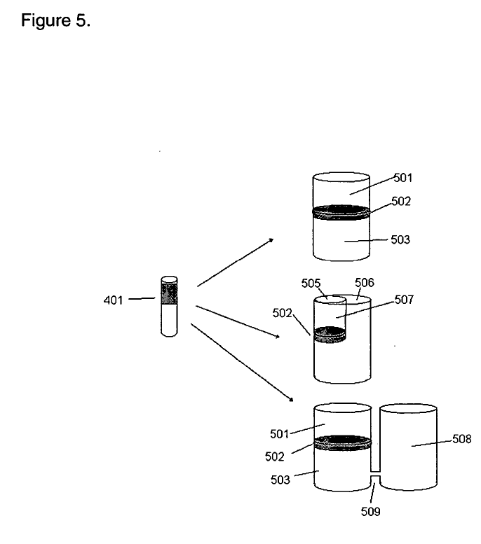

FIG. 5 displays some embodiments of container 401. In some embodiments, the air space 501 and 503 can be separated by filter 502. Sample can first enter air space 501 and filtrate can enter air space 503. Pipettor 111 can pierce filter 502 to contact the filtrate. In some embodiments, sample can first enter in air space 507 via opening 505, can be filtered by filter 502 and the filtrate can enter air space 506. Pipettor 111 can then contact the filtrate without contacting filter 502 via opening 506. The bottom of the container can be arranged in such as way to enable pipettor 111 to aspirate almost all of the filtrate. In some embodiments, air space 507 and filter 502 can be annular rings while opening 506 can be in the center of the annulus. In this and similar embodiments, the radial symmetry of container 401 can make placement in container-holder 402 rotation-independent. In some embodiments, sample can first enter air space 501 and filtrate can enter air space 503 and then flow into air space 508 via connector 509. Pipettor 111 can then contact the filtrate without contacting filter 502. In embodiments where container 401 is not radially symmetric, container-holder 402 can be keyed to ensure the detection system knows the orientation of container 401. To accelerate the filtration process, container-holder 402 can operate as a centrifuge to increase the gravitational force (via the theory of general relativity) or the portion of the container top that receives the sample may be sealed and pressurized. Topological equivalents of these containers 401 are also part of the invention.

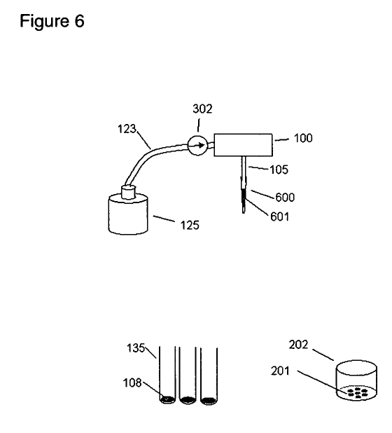

As shown is FIG. 6, some embodiments of the invention use pipette tip 600 that contains a filter 601. Filtrate from sample collection device 100 can pass through tubing 105 and pipette tip 600 into binding reagent container 135 or binding reagent container 202, depending on the detection system. Optionally, the sample collection device can use extraction buffer 115 via tubing 123.

Some embodiments can be variations on FIG. 4 and/or FIG. 6. Rather than having pump 302 in tubing 123, the pump can be placed in tubing 105 in a similar manner to pump 301. The pair of one-way valves 102 and 115 can optionally be present, for example, as a means to empty sample collection device 100.

In certain embodiments, filter 107, 502, and/or 601 have the properties and compositions described elsewhere in this specification. In certain embodiments, the extraction buffer located in reagent bottle 125 has the properties and compositions described elsewhere in this specification.

Pumps 113, 301, and 302 can be of several types. For example, the pumps can be that described in US patent publication US 2004-0096368 A1, or PCT WO 2005/114175A2. The pumps can be peristaltic, syringe, gear, piston (including the valveless designs sold by Fluid Metering, Inc., Syosset, N.Y. as well as the more traditional designs employing valves), or other positive-displacement designs. The pumps can be pressure sources, such as vacuum pumps and impeller pumps. In certain embodiments, the pumps in this invention can not solely rely on capillary action to move liquids.

IV. KITS

In some embodiments, the present invention provides a kit comprising reagents for performing an assay to detect an analyte of interest, a filter for filtering a sample, and at least one extraction buffer for reducing the effect of the sample matrix on the assay.

In certain embodiments, the invention provides a kit for performing the above methods, comprising a filter and at least one reagent selected from (a) an extraction buffer that has a pH>8 or pH<6 and an osmolarity greater than or equal to 0.1 osmol/L and (b) an extraction buffer that has an osmolarity greater than 1.1 osmol/L. In some embodiments, the kit further comprises a labeled binding partner specific to said analyte. In some embodiments, the kit further comprises a labeled first binding partner and a second binding partner specific to said analyte. In some embodiments, at least one of the first and second binding partners is an antibody.

In some embodiments, the kit further comprises a labeled analog of the analyte and a binding partner specific to said analyte. In some embodiments, the labeled analog of the analyte is a labeled known amount of the analyte itself.

In certain embodiments, the reagents for performing the assay can be provided in a binding reagent container as described above, while the filter and at least one extraction buffer for reducing the effect of the sample matrix on the assay are provided in a second container.

In various embodiments, the kit can provide written instructions in the form of an insert or packaging that describe how to use the kit.

In certain kits according to the invention, the reagents for performing the assay comprise at least one of the labels discussed above.

In some embodiments of the invention, the reagents for performing the assay comprise at least one of the supports discussed above.

The kit compositions can comprise any reagents useful for performing an assay to detect an analyte of interest. In certain embodiments, the kits can comprise any of containers, humidity indicators, or humidity barriers for use in the assay or to maintain the integrity of the reagents.

The following examples are meant to be illustrative of the invention and not in any way limiting of the invention's scope and application. The skilled artisan can recognize additional embodiments reflective of the invention's goals.

### EXAMPLES

These examples describe the beneficial effects of treating samples before or during an analyte detection immunoassay. Without such treatment, components in the sample matrix can interfere with binding interactions needed to detect a particular analyte. As a result, the binding signal and/or the signal to background ratios (S:B) can be reduced, making detection of the analyte difficult. Reagents and other definitions described in one example also apply to the other examples unless explicitly superseded by a description/definition in that example.

### Example 1

### Environmental Matrix Significantly Reduces Signal and Signal to Background Ratios

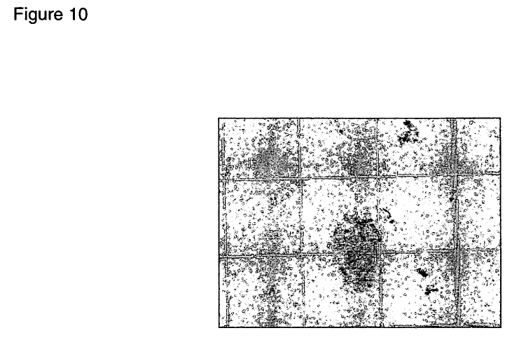

An immunoassay was performed using a mouse IgG antibody as the analyte to be detected. Analyte standards were prepared by diluting the stock mouse IgG to 10 μg/ml in either a matrix prepared by filtering the air in a mailroom through a wet concentrator (MR matrix) or in phosphate-buffered saline (PBS matrix) to determine the effect that an environmental matrix (e.g., the MR matrix) can have on assays. PBS matrix contained 0.3% (v/v) Tween® 20 (polyoxyethylene sorbitan monolaurate), 10 mM phosphate buffer, 120 mM NaCl, 2.7 mM KCl, 0.1% (v/v) hydroxypyridine, and 0.1% (v/v) methylisothiazolinone (MIT) at a pH of 7.4. Materials present in the MR matrix are shown in FIG. 10. The diluted stock solutions were further serially diluted either with MR matrix or with PBS matrix to yield samples with concentrations mouse IgG ranging from 0.5 ng/ml to 1000 ng/ml.

Beads coated with goat anti-mouse IgG Fc antibodies were prepared, as follows. First, the goat antibodies were labeled with biotin. A biotin solution was prepared by diluting NHS-LC-LC-Biotin (Pierce) in dimethylsulfoxide (DMSO) to a concentration of 1 mg/ml. A volume containing 0.5 mg of the goat antibody in PBS was mixed with 100 μl of the biotin solution. The mixture was incubated for 1 hour at room temperature with end over end shaking. The goat antibody-biotin conjugate was dialyzed against three changes of PBS, changing buffer every 5 hours, using Pierce slide-a-lyzers. Streptavidin coated 2.8 μm diameter superparamagnetic beads (Dynal Biotech), 4 mg, were combined with 10 μg of biotin-labeled goat antibodies and incubated for 1 hour at room temperature in a microfuge tube with end over end shaking. The beads were washed 3 times with 1 ml of assay buffer and reconstituted to a final concentration of 5 mg/ml.

Anti-mouse IgG F(ab)₂ fragments labeled with [Ru(bpy)₃]²⁺ (BV-TAG NHS Ester, BioVeris Corp, Gaithersburg, Md.) according to the manufacturer's instructions.

The immunoassay was performed by diluting the antibody coated beads 1:40 in assay buffer and adding 25 μl of this suspension to each assay well. Fifty microliters of each mouse IgG sample was added to each well, in triplicate. [Ru(bpy)₃]²⁺ labeled F(ab)₂ fragments were diluted to 670 ng/ml in assay buffer. Twenty-five microliters of the labeled F(ab)₂ fragment solution was added to all assay wells. The assay plate was incubated at room temperature with shaking for 15 minutes Three hundred microliters of PBS diluent was added to all wells prior to measuring the amount of the [Ru(bpy)₃]²⁺-labeled F(ab)₂ fragment bound to the superparamagnetic beads using an M-Series M1M Analyzer (BioVeris Corp.) according to the manufacturer's instructions.

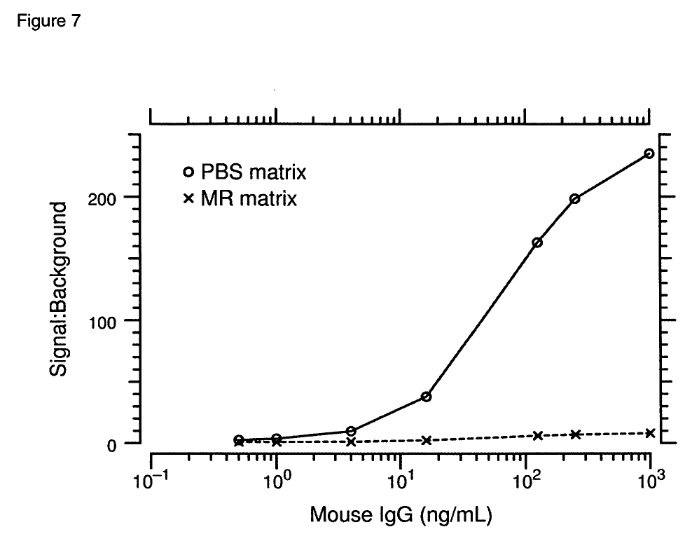

The results are shown in Table 3 and in FIG. 7. The data show that the MR matrix significantly reduced the signal to background ratio in the assay.

TABLE 3

| | Mean ECL | Mean ECL | | |
|---|---|---|---|---|
| Mouse IgG | Signal PBS | Signal | Mean S:B | Mean S:B |
| ng/ml | Matrix | MR Matrix | PBS Matrix | MR Matrix |
| 1000 | 68947 | 42621 | 234.75 | 8.00 |
| 250 | 58295 | 37686 | 198.48 | 7.07 |
| 125 | 47887 | 32464 | 163.05 | 6.09 |
| 16 | 11151 | 13009 | 37.97 | 2.44 |
| 4 | 2893 | 6963 | 9.85 | 1.31 |
| 1 | 1115 | 6032 | 3.80 | 1.13 |
| 0.5 | 757 | 5597 | 2.58 | 1.05 |
| 0 | 294 | 5330 | 1.00 | 1.00 |

### Example 2

### Effect of Sample Preparation on Signal and S:B Ratio

Samples of mouse IgG diluted with MR matrix were prepared as described in Example 1. An immunoassay was performed by diluting the anti-mouse IgG Fc fragment antibody-coated beads 1:40 in assay buffer and adding 25 μl of this suspension to each assay well. Fifty microliters of each mouse IgG sample was added to each well, in duplicate. [Ru(bpy)₃]²⁺ labeled F(ab)₂ fragments were diluted to 670 ng/ml in assay buffer. Twenty-five microliters of the labeled F(ab)₂ fragment solution was added to all assay wells. The assay plate was incubated at room temperature with shaking for 15 minutes Three hundred microliters of borate buffer (0.1 M sodium borate, 0.5M NaCl, 0.3% Tween®20, pH 8.5) was added to all wells prior to measuring the amount of the [Ru(bpy)₃]²⁺-labeled F(ab)₂ fragment bound to the superparamagnetic beads using an M-Series M1M Analyzer (BioVeris Corp.) according to the manufacturer's instructions. PBS diluent uses different preservatives than PBS matrix: PBS diluent comprises 0.1% (v/v) Kathon II cG/ICP (Sigma Aldrich) instead of the hydroxypyridine and MT.

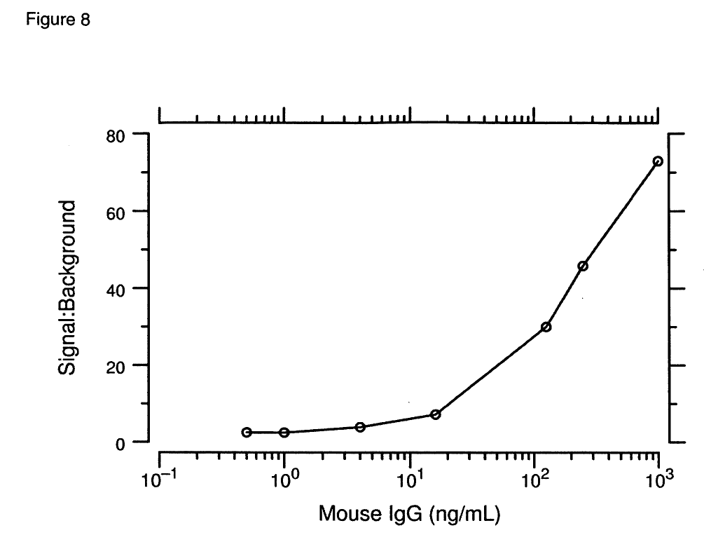

The results are shown in Table 4 and FIG. 8. By comparison to Table 3 and FIG. 7, it can be seen that adding borate buffer to the immunoassay after the incubation, but prior to the ECL measurement significantly increases the signal to background ratio.

TABLE 4

| Mouse IgG | Mean ECL | |
|---|---|---|
| ng/ml | Signal | Mean S:B Ratio |
| 1000 | 53268 | 72.94 |
| 250 | 33473 | 45.83 |
| 125 | 21916 | 30.01 |
| 16 | 5270 | 7.22 |
| 4 | 2869 | 3.93 |
| 1 | 1817 | 2.49 |
| 0.5 | 1814 | 2.52 |
| 0 | 730 | 1.00 |

### Example 3

### Effect of Matrix Filtration

An immunoassay to detect ricin was performed on samples diluted with MR matrix. Ricin (Ricinus communis agglutinin II, RCA₆₀) was diluted with MR matrix to concentration of 1,100 ng/ml, 10 ng/ml, and 1 ng/ml. Prior to the assay, a portion of each sample was filtered through a 5 μm Acrodisc® syringe filter (Pall Corp., Elmhurst, N.Y.) using a 5 ml syringe. An antibody that binds to ricin was used to coat beads as described for Example 1. A second antibody that binds ricin was labeled with [Ru(bpy)₃]²⁺ as described in Example 1.

Twenty-five microliters of antibody-coated superparamagnetic bead suspension was added to each assay well. Fifty microliters of the each filtered ricin sample was added to assay wells, in duplicate. To compare the effect of MR matrix dilution with filtration, 100 μl of unfiltered ricin sample was added to assay wells. Twenty five microliters of [Ru(bpy)₃]²⁺⁻ labeled anti-ricin antibody and twenty-five microliters of the biotin labeled antibody coated beads were added to all assay wells. The assay plate was incubated at room temperature with shaking for 15 minutes Three hundred microliters of borate buffer (0.1 M sodium borate, 0.5M NaCl, 0.3% Tween® 20, pH 8.5) was added to all wells prior to measuring the amount of the [Ru(bpy)₃]²⁺-labeled F(ab)₂ fragment bound to the superparamagnetic beads using an M-Series M1M Analyzer (BioVeris Corp.) according to the manufacturer's instructions.

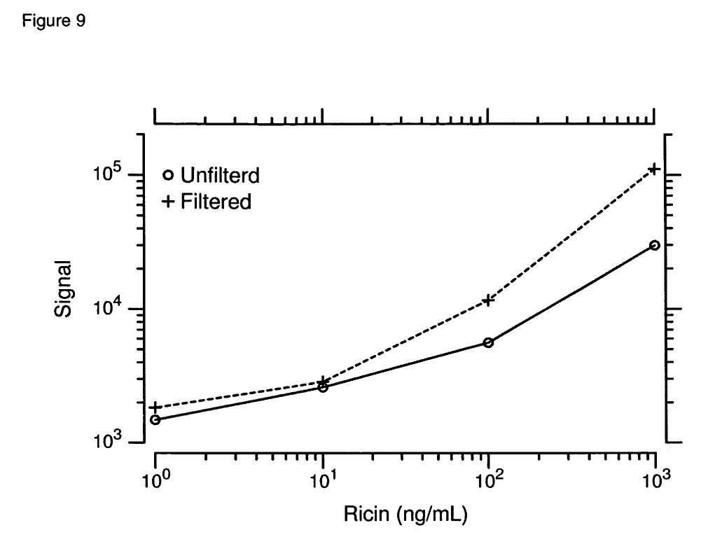

As shown in Table 5 and FIG. 9, filtering the analyte sample substantially improved the signal and the signal to background ratio in comparison to unfiltered samples.

TABLE 5

| | | | Sample | | | | | |
|---|---|---|---|---|---|---|---|---|
| Ricin | | | volume, | | | | | |
| ng/ml | Matrix | Diluent | filter status | Rep. 1 | Rep. 2 | Mean | % CV | S:B |
| 0 | MR | Borate | 50 μl, filtered | 2369 | 10394* | 2369 | | 1 |
| 1000 | MR | Borate | 50 μl, filtered | 114142 | 106720 | 110431 | 5% | 46.62 |
| 100 | MR | Borate | 50 μl, filtered | 11765 | 11559 | 11662 | 1% | 4.92 |
| 10 | MR | Borate | 50 μl, filtered | 2613 | 3111 | 2862 | 12% | 1.21 |
| 1 | MR | Borate | 50 μl, filtered | 1830 | 1832 | 1831 | 0% | 0.77 |
| 0 | MR | Borate | 100 μl, unfiltered | 1239 | 1906 | 1573 | 30% | 1 |
| 1000 | MR | Borate | 100 μl, unfiltered | 31958 | 27539 | 29749 | 11% | 18.92 |
| 100 | MR | Borate | 100 μl, unfiltered | 4615 | 6608 | 5612 | 25% | 3.57 |
| 10 | MR | Borate | 100 μl, unfiltered | 2329 | 2876 | 2603 | 15% | 1.66 |
| 1 | MR | Borate | 100 μl, unfiltered | 1495 | 1469 | 1482 | 1% | 0.94 |

*This value was discarded.

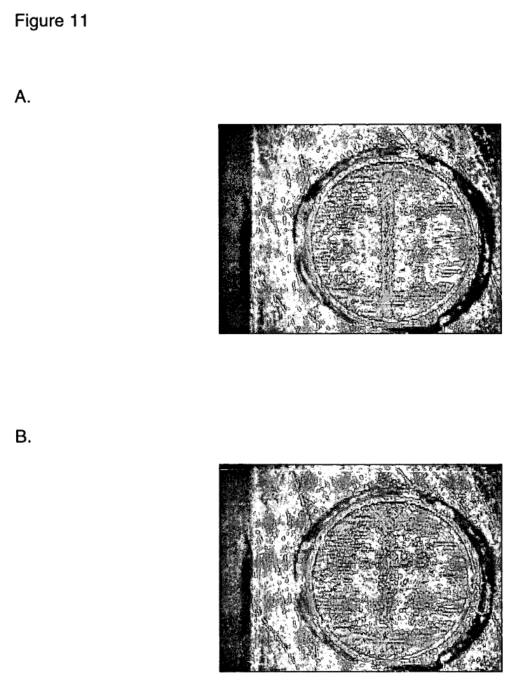

FIG. 11 shows the difference between the deposition of superparamagnetic beads from filtered and unfiltered samples on the measuring cell electrode. The innermost circle is the portion of the working electrode that is directly visible to the photodetector, which has been removed to obtain these images. The brown specks that form one or more vertical bands are the superparamagnetic beads. (A) shows the deposition of beads when the sample was processed with a filter and an extraction buffer according to the invention. (B) is a photograph of bead deposition starting with the same MR matrix-containing sample that was processed without filtering. The images show that the matrix affects the number of beads captured as well as their distribution, both of which are expected to affect the measured electrochemiluminescence.

### Example 4

### Assay Sensitivity

To test the lower limits of sensitivity of the ricin assay performed according to the invention, ricin was diluted in MR matrix to 0.2, 2, 20, 200, and 2000 pg/ml. The samples were filtered and assayed as described in Example 3, except that no borate buffer was added until after the immunoassay incubation step was complete, and that Critical Reagent Program (CRP) reagents supplied by the United States government were used. Measurements were done in duplicate, except for 0 pg/ml concentration which had a single measurement.

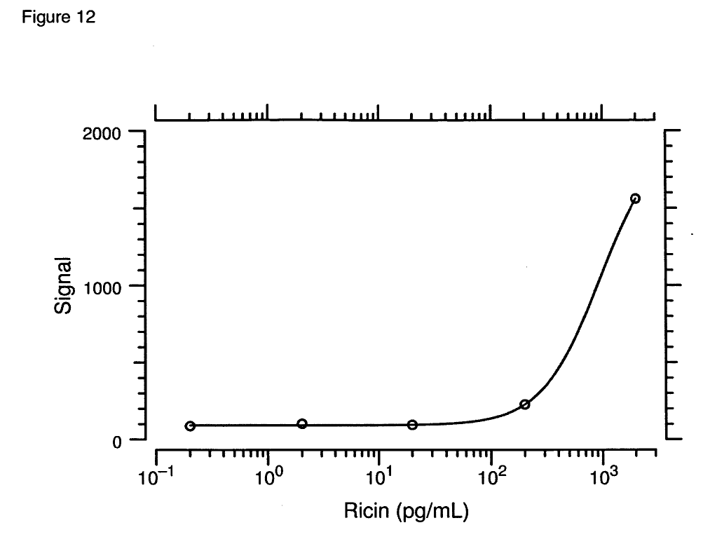

Table 6 shows the signal to background ratios for the experiment. FIG. 12 shows the signal values for the experiment. The assay was sensitive to at ricin concentrations of least 200 pg/ml.

TABLE 6

| | | Sample | | Mean | % | |
|---|---|---|---|---|---|---|
| Ricin | | Vol. | Test Kit | ECL | CV | S/B |
| 0 | | 50 μl | CRP Reagents | 89 | N/A | 1 |
| 2000 | pg/ml | 50 μl | CRP Reagents | 1561 | 14% | 17.53 |
| 200 | pg/ml | 50 μl | CRP Reagents | 228 | 6% | 2.56 |
| 20 | pg/ml | 50 μl | CRP Reagents | 95 | 1% | 1.06 |
| 2 | pg/ml | 50 μl | CRP Reagents | 102 | 14% | 1.15 |
| 0.2 | pg/ml | 50 μl | CRP Reagents | 87 | 3% | 0.98 |

### Example 5

### Effect of Borate Buffer Alone on Assay Signal

To determine whether borate buffer improved assay performance, anthrax spores (Sterne strain) were diluted in PBS matrix or in a 50%/50% mixture of MR matrix and borate buffer to concentrations that ranged from 10² colony-forming units (cfu)/ml to 10⁶ cfu/ml. Aliquots of each sample were assayed for the spores without filtration using standard CRP reagents according to the manufacturer's instructions. Briefly, 1001 μl of the sample filtrate or PBS diluted spore were added to each tube containing the CRP assay reagents. The tubes were then placed in the M1M Analyzer and set to incubate and read. At the end of the 15 minute incubation, 300 μl of PBS diluent was added to all the samples. The amount of [Ru(bpy)₃]²⁺-labeled anti-anthrax antibody bound to the superparamagnetic beads was measuring using an M1M Analyzer (BioVeris Corp.) according to the manufacturer's instructions.

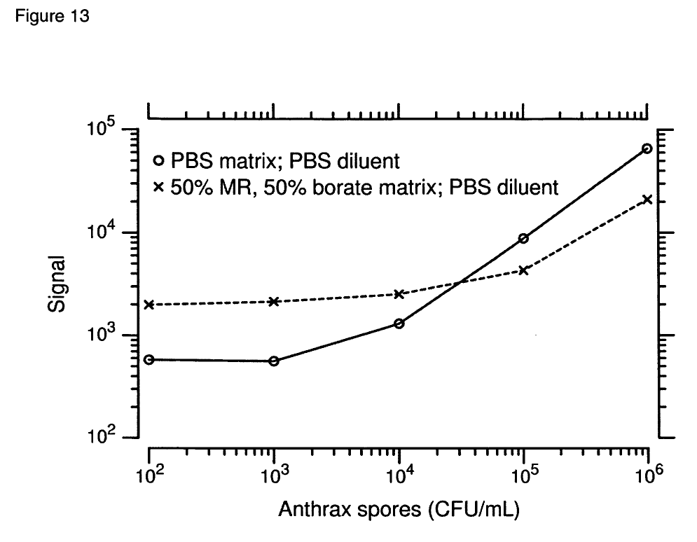

Table 7 and FIG. 13 show the results. The signal and the signal to background ratio measured in the presence of MR matrix and borate buffer in samples that had not been filtered were both decreased over control values.

TABLE 7

| Anthrax | | | | | |
|---|---|---|---|---|---|
| spores | | | Mean ECL | | |
| CFU/ml | Matrix | Diluent | Signal | % CV | S:B |
| 0 | 50% MR, | PBS | 1840 | 5% | 1 |
| | 50% Borate | | | | |
| 1 × 10² cfu/ml | 50% MR, | PBS | 1989 | 2% | 1.08 |
| | 50% Borate | | | | |
| 1 × 10³ cfu/ml | 50% MR, | PBS | 2133 | 4% | 1.16 |
| | 50% Borate | | | | |
| 1 × 10⁴ cfu/ml | 50% MR, | PBS | 2525 | 3% | 1.37 |
| | 50% Borate | | | | |
| 1 × 10⁵ cfu/ml | 50% MR, | PBS | 4313 | 2% | 2.34 |
| | 50% Borate | | | | |
| 1 × 10⁶ cfu/ml | 50% MR, | PBS | 21079 | 3% | 11.46 |
| | 50% Borate | | | | |
| 0 | PBS | PBS | 504 | 5% | 1 |
| 1 × 10² cfu/ml | PBS | PBS | 579 | 25% | 1.15 |
| 1 × 10³ cfu/ml | PBS | PBS | 561 | 7% | 1.11 |
| 1 × 10⁴ cfu/ml | PBS | PBS | 1303 | 2% | 2.58 |
| 1 × 10⁵ cfu/ml | PBS | PBS | 8807 | 3% | 17.47 |
| 1 × 10⁶ cfu/ml | PBS | PBS | 65763 | 1% | 130.48 |

An additional experiment was run wherein the borate buffer was used instead of PBS diluent in the 300 μl dilution after the incubation period. Results (not shown) were similar to Table 7 and FIG. 13. Adding borate buffer after the incubation period did not affect the results without filtration.

### Example 6

### The Effect of Borate Buffer Added Before Filtration

To determine whether the addition of borate buffer to environmental matrix samples prior to the filtration step would affect the later-measured ECL signal, the following experiment was performed. Two sets of samples containing Bacillus globigii (BG) were prepared: serial dilutions of BG in MR matrix diluted 1:1 with borate buffer (“sample 1”); and serial dilutions of BG in MR matrix alone (“sample 2”). Both sets of samples were filtered using a 5 μm Acrodisc® syringe filter as described above.

Twenty-five microliters of anti-BG antibody-coated superparamagnetic bead suspension was added to each assay well. One hundred microliters of the sample 1 dilutions was added in triplicate to assay wells. Fifty microliters of each sample 2 dilution was added to 3 assay wells. Twenty-five microliters [Ru(bpy)₃]²⁺-labeled anti-BG antibody were added to all assay wells. The assay plate was incubated at room temperature with shaking for 15 minutes. Three hundred microliters of borate buffer was added then to each of the sample 1 wells and to three of each set of six sample 2 wells. Three hundred microliters of PBS diluent was added to the remaining sample 2 wells. The amount of the [Ru(bpy)₃]²⁺-labeled antibody bound to the superparamagnetic beads was then measured using an M-Series M1M Analyzer (BioVeris Corp.) according to the manufacturer's instructions.

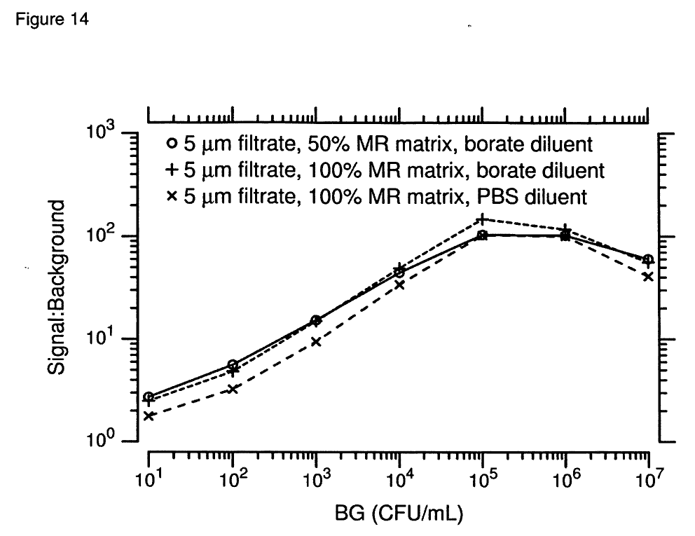

Table 8 and FIG. 14 show the results. The signal and the signal to background ratio at low analyte concentrations was improved by having the borate diluent.

TABLE 8

| | 50% MR Matrix | | 100% MR Matrix | | 100% MR Matrix | |
|---|---|---|---|---|---|---|
| | Borate Diluent | | Borate Diluent | | PBS diluent | |
| BG | Mean ECL Signal | | Mean ECL Signal | | Mean ECL Signal | |
| CFU/ml | [S:B] | | [S:B] | | [S:B] | |
| 0 | 610 | [1.00] | 854 | [1.00] | 1820 | [1.00] |
| 1 × 10¹ | 1666 | [2.73] | 2136 | [2.50] | 3235 | [1.78] |
| 1 × 10² | 3433 | [5.63] | 4147 | [4.85] | 5930 | [3.26] |
| 1 × 10³ | 9395 | [15.40] | 12856 | [15.05] | 17257 | [9.48] |
| 1 × 10⁴ | 27251 | [44.67] | 42209 | [49.40] | 62196 | [34.17] |
| 1 × 10⁵ | 63335 | [103.83] | 126069 | [147.54] | 186969 | [102.71] |
| 1 × 10⁶ | 62800 | [102.95] | 100951 | [118.15] | 183958 | [101.06] |
| 1 × 10⁷ | 36962 | [60.59] | 48068 | [56.26] | 74875 | [41.13] |

The specification is most thoroughly understood in light of the teachings of the references cited within the specification. The embodiments within the specification provide an illustration of embodiments of the invention and should not be construed to limit the scope of the invention. The skilled artisan readily recognizes that many other embodiments are encompassed by the invention. All publications and patents cited in this disclosure are incorporated by reference in their entirety. To the extent the material incorporated by reference contradicts or is inconsistent with this specification, the specification will supercede any such material. The citation of any references herein is not an admission that such references are prior art to the present invention.

Unless otherwise indicated, all numbers expressing quantities of ingredients, reaction conditions, and so forth used in the specification, including claims, are to be understood as being modified in all instances by the term “about.” Accordingly, unless otherwise indicated to the contrary, the numerical parameters are approximations and may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the following claims.

1. A method of analyzing a sample suspected of containing an analyte comprising:
(a) filtering the sample through a filter;
(b) adding a first labeled binding partner specific to said analyte;
(c) optionally adding a second binding partner specific to said analyte;
(d) adding an extraction buffer chosen from:
(i) an extraction buffer that has a pH≧8 or pH≦6 and an osmolarity greater than or equal to 0.1 osmol/L or
(ii) an extraction buffer that has an osmolarity greater than 1.1 osmol/L; and
(e) measuring the amount of the labeled first binding partner in complexes formed between the first binding partner, the analyte, and, if present, the second binding partner,
wherein steps (a) through (d) may be performed in any order and step (e) is performed last.

2. (canceled)

3. The method of claim 1, wherein the filter has a pore size rating less than or equal to about 10 μm and greater than or equal to about 1 μm.

4-5. (canceled)

6. The method of claim 1, wherein the extraction buffer has a pH ? 8 and an osmolarity greater than or equal to 0.8 osmol/L.

7-8. (canceled)

9. The method of any one of claims 1, 3, and 6, wherein the binding partner is labeled with an electrochemiluminescent (ECL) moiety.

10-18. (canceled)

19. The method of claim 1, wherein the method is a method for analyzing the sample for multiple analytes.

20-25. (canceled)

26. A method of analyzing a sample suspected of containing an analyte comprising:
(a) filtering the sample through a filter;
(b) adding a labeled analog of the analyte
(c) adding a binding partner specific to said analyte;
(d) adding an extraction buffer chosen from:
(i) an extraction buffer that has a pH≧8 or pH≦6 and an osmolarity greater than or equal to 0.1 osmol/L and
(ii) an extraction buffer that has an osmolarity greater than 1.1 osmol/L; and
(e) measuring the amount of the labeled analog of the analyte bound to the binding partner,
wherein steps (a) through (d) may be performed in any order and step (e) is performed last.

27. (canceled)

28. The method of claim 26, wherein the filter has a pore size rating less than or equal to about 10 μm and greater than or equal to about 1 μm.

29-30. (canceled)

31. The method of claim 26, wherein the extraction buffer has a pH≧8 and an osmolarity greater than or equal to 0.8 osmol/L.

32-33. (canceled)

34. The method of any one of claims 26, 28, and 31, wherein the binding partner is labeled with an electrochemiluminescent (ECL) moiety.

35-49. (canceled)

50. A kit for performing the method of claim 1 comprising a filter and at least one reagent selected from (a) an extraction buffer that has a pH≧8 or pH≦6 and an osmolarity greater than or equal to 0.1 osmol/L and (b) an extraction buffer that has an osmolarity greater than 1.1 osmol/L.

51. The kit of claim 50, wherein said extraction buffer has a pH>8 and an osmolarity greater than or equal to 0.8 osmol/L.

52-56. (canceled)

57. A kit for performing the method of claim 26, comprising a filter and at least one reagent selected from (a) an extraction buffer that has a pH≧8 or pH≦6 and an osmolarity greater than or equal to 0.1 osmol/L and (b) an extraction buffer that has an osmolarity greater than 1.1 osmol/L.

58-63. (canceled)

64. An apparatus for use in analyzing sample suspected of containing one or more analytes comprising:
(a) a filter fluidically connected to a sampling device via a first one-way valve wherein the first one-way valve is oriented to allow flow from the sampling device to the filter;
(b) a waste line fluidically connected to a second one-way valve that is fluidically connected to a point fluidically between the first one-way valve and the filter where the second one-way valve is oriented to allow flow from the filter to the waste line;
(c) a pump arranged to be able to reversibly drive liquid flow across the filter;
(d) a binding reagent container that holds a labeled binding partner specific for each of said one or more analytes;
(e) means for depositing filtrate from said filter into said binding reagent container; and
(f) means for measuring the labeled binding partner.

65. An apparatus for use in analyzing sample suspected of containing one or more analytes comprising:
(a) a filter fluidically connected to a sampling device;
(b) a pump arranged to be able to drive liquid flow across the filter;
(c) a binding reagent container that holds a labeled binding partner specific for each of said one or more analytes;
(d) means for depositing filtrate from said filter into said binding reagent container; and
(e) means for measuring the labeled binding partner,
wherein at least one of the following elements applies: (i) the apparatus further comprises at least one electrode; (ii) the pump does not solely rely on capillary action to move liquids; (iii) the pump is a positive displacement pump; (iv) the apparatus further comprises a means for adding an extraction buffer to the sample; (v) the apparatus further comprises a means for adding an extraction buffer to the sample before the sample contacts the filter; and (vi) the sampling device is an air sampler.

66. The apparatus of claim 64 or 65 additionally comprising
(a) at least one working electrode for initiating electrochemiluminescence; and
(b) a photodetector.

67-75. (canceled)

76. A sample filtering device comprising
(a) an upper container that is fluidically sealed from a lower container by a filter; and
(b) an opening where the lower container can be accessed without going through the upper container.

77. The sample filtering device of claim 76, wherein the device has radial symmetry.

78-79. (canceled)

80. The apparatus of any one of claims 64 or 65, wherein the filter has a pore size rating less than or equal to about 10 μm and greater than or equal to about 1 μm.

81-82. (canceled)
